(19) United States
(12) Patent Application Publication
(10) Pub. No.: US 2015/0179954 A1
(43) Pub. Date: Jun. 25, 2015

(54) SUBSTITUTED TERRYLENE AND QUATERRYLENE DERIVATES AND USE AS SEMICONDUCTORS THEREOF

(71) Applicants: BASF SE, Ludwigshafen (DE); MAX-PLANCK-GESELLSCHAFT ZUR FOERDERUNG DER WISSENSCHAFTEN E.V., Muenchen (DE)

(72) Inventors: Thomas Gessner, Heidelberg (DE); Helmut Reichelt, Neustadt (DE); Glauco Battagliarin, Mannheim (DE); Chen Li, Koeln (DE); Klaus Muellen, Koeln (DE)

(73) Assignees: BASF SE, Ludwigshafen (DE); MAX-PLANCK-GESELLSCHAFT ZUR FOERDERUNG DER WISSENSCHAFTEN E.V., Muenchen (DE)

(21) Appl. No.: 14/411,184

(22) PCT Filed: Jun. 28, 2013
(86) PCT No.: PCT/IB13/55309

§ 371 Date: Dec. 24, 2014

Related U.S. Application Data
(60) Provisional application No. 61/665,914, filed on Jun. 29, 2012.

(30) Foreign Application Priority Data
Jun. 29, 2012 (EP) 12174337.1

(51) Int. Cl. H01L 51/00; C07D 487/06; C07F 5/04

(52) U.S. Cl. CPC H01L 51/0072; C07F 5/04; C07D 487/06; H01L 51/0545

(57) ABSTRACT

Disclosed are terrylene and quaterrylene derivates of general formula (I) and the use thereof as organic semiconductor materials.

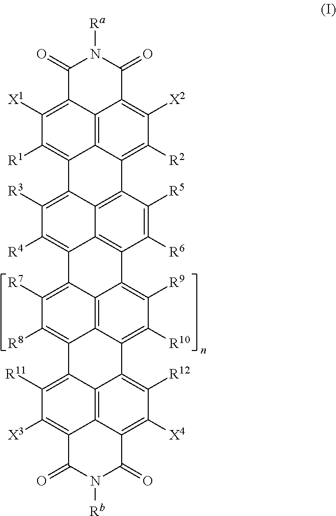

### FIELD OF THE INVENTION

It is expected that, in the future, not only the classical inorganic semiconductors but increasingly also organic semiconductors based on low molecular weight or polymeric materials will be used in many sectors of the electronics industry. In many cases, these organic semiconductors have advantages over the classical inorganic semiconductors, for example better substrate compatibility and better processability of the semiconductor components based on them. They allow processing on flexible substrates and enable their interface orbital energies to be adjusted precisely to the particular application range by the methods of molecular modeling. The significantly reduced costs of such components have brought a renaissance to the field of research of organic electronics.

Organic electronics is concerned principally with the development of new materials and manufacturing processes for the production of electronic components based on organic semiconductor layers. These include in particular organic field-effect transistors (OFETs) and organic electroluminescent devices (hereinafter abbreviated as “EL” devices). Great potential for development is ascribed to organic field-effect transistors, for example in storage elements and integrated optoelectronic devices. An organic electroluminescent device is a self-emission device utilizing the principle that a fluorescent material emits light by the recombination energy of holes injected from an anode and electrons injected from a cathode when an electric field is applied. EL devices in form of organic light-emitting diodes (OLEDs) are especially of interest as an alternative to cathode ray tubes and liquid-crystal displays for producing flat visual display units. Owing to the very compact design and the intrinsically low power consumption, devices which comprise OLEDs are suitable especially for mobile applications, for example for applications in cell phones, laptops, etc. In addition, OLEDs offer great advantages over the illumination technologies known to date, especially a particularly high efficiency.

Organic photovoltaics is concerned principally with the development of new materials for organic solar cells. A great potential for development is ascribed to materials which have maximum transport widths and high mobilities for light-induced excited states (high exciton diffusion lengths) and are thus advantageously suitable for use as an active material in so-called excitonic solar cells. It is generally possible with solar cells based on such materials to achieve very good quantum yields. There is therefore a great need for organic compounds which are suitable as charge transport materials or exciton transport materials.

R. Schmidt, J. H. Oh, Y.-S. Sun, M. Deppisch, A.-M. Krause, K. Radacki, H. Braunschweig, M. Könemann, P. Erk, Z. Bao and F. Würthner J. Am. Chem. Soc. 2009, 131, 6215-6228 describes halogenated perylene bisimide derivatives, for example

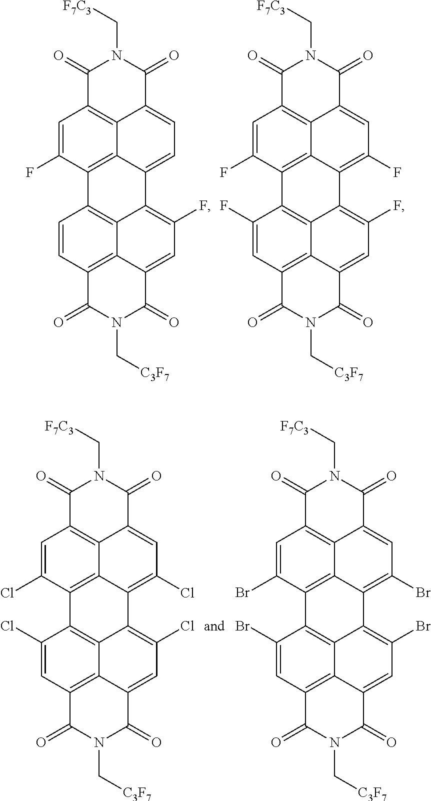

S. Nakazono, Y. Imazaki, H. Yoo, J. Yang, T. Sasamori, N. Tokitoh, T. Cedric, H. Kageyama, D. Kim, H. Shinokubo and A. Osuka Chem. Eur. J. 2009, 15, 7530-7533 describes the preparation of 2,5,8,11 tetraalkylated perylene tetracarboxylic acid bisimides from perylene tetracarboxylic acid bisimides

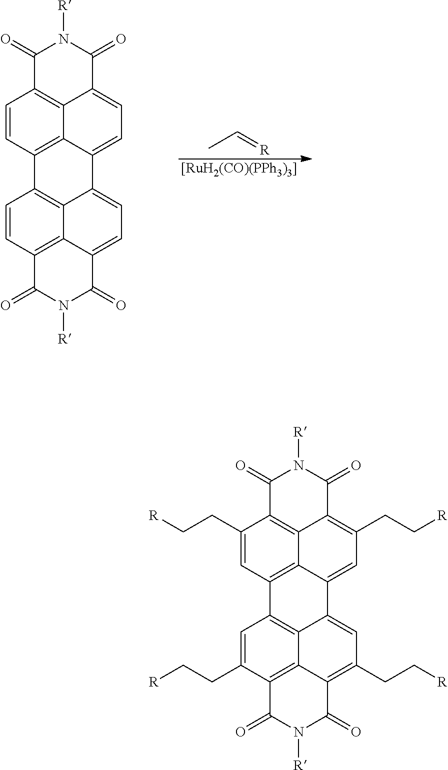

S. Nakanzono, S. Easwaramoorthi, D. Kim, H. Shinokubo, A. Osuka Org. Lett. 2009, 11, 5426 to 5429 describes the preparation of 2,5,8,11 tetraarylated perylene tetracarboxylic acid bisimides from perylene tetracarboxylic acid bisimides

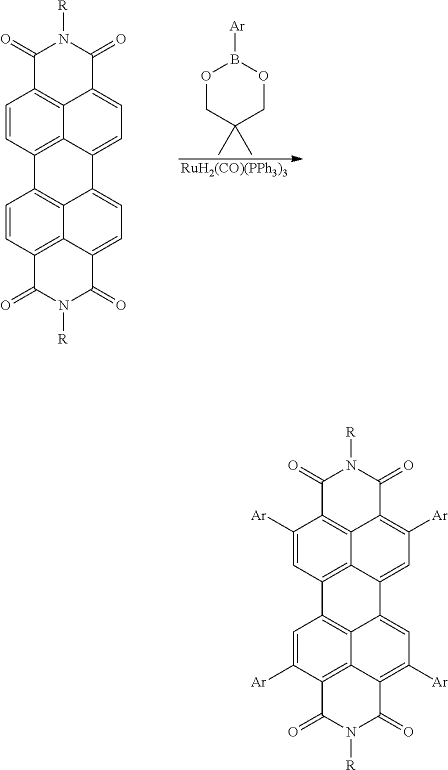

U.S. Pat. No. 7,355,198 B2 describes an organic thin film transistor (OFET), which interposes an organic acceptor film between source and drain electrodes and an organic semiconductor film. The organic semiconductor film is formed of pentacene. In particular, the organic acceptor film is formed of at least one electron withdrawing material selected from a long list of compounds, including N,N′-bis(di-tert-butyphenyl)-3,4,9,10-perylenedicarboximide.

U.S. Pat. No. 7,326,956 B2 describes a thin film transistor comprising a layer of organic semiconductor material comprising tetracarboxylic diimide perylene-based compound having attached to each of the imide nitrogen atoms a carbocyclic or heterocyclic aromatic ring system substituted with one or more fluorine containing groups. In one embodiment the fluorine-containing N,N′-diaryl perylene-based tetracarboxylic diimide compound is represented by the following structure:

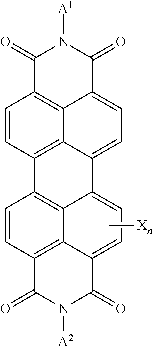

wherein A¹ and A² are independently carbocyclic and/or heterocyclic aromatic ring systems comprising at least one aromatic ring in which one or more hydrogen atoms are substituted with at least one fluorine-containing group. The perylene nucleus can be optionally substituted with up to eight independently selected X groups, wherein n is an integer from 0 to 8. The X substituent groups on the perylene can include a long list of substituents, including halogens such as fluorine or chlorine.

WO 2007/093643 describes fluorinated rylenetetracarboxylic acid derivatives. Preferred compounds are of formula IBa

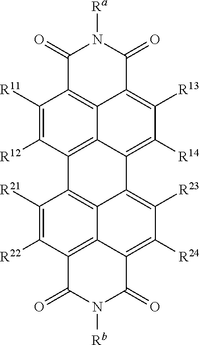

wherein 1, 2, 3, 4, 5 or 6 of the residues R¹¹, R¹², R¹³, R¹⁴, R²¹, R²², R²³ and R²⁴ are F, optionally at least one of the residues R¹¹, R¹², R¹³, R¹⁴, R²¹, R²², R²³ and R²⁴, which is not F, can independently be Cl or Br, and the remaining residues are H, and

R^{a} and R^{b} are independently from each other are H or an organic residue.

WO 2006/117383 A1 describes terrylene and quaterrylene derivatives and their use as pigments and IR dyes.

WO 2008/063609 describes a compound having the following formula

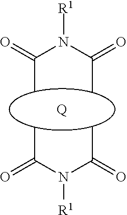

wherein Q can be

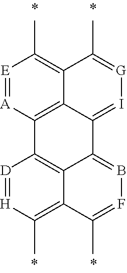

wherein A, B, I, D, E, F, G and H are independently selected from a group of substituents, including, CH and CR^{a}, wherein R^{a} can be selected from a list of substituents, including halogen. For example, A, B, I, D, E, F, G and H can be independently CH, C—Br or C—CN.

WO 2009/024512 describes halogen-containing perylenetetracarboxylic acid derivatives, and in particular compound IBa

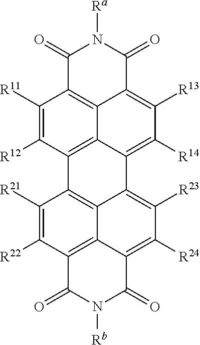

wherein the residues R¹¹, R¹², R¹³, R¹⁴, R²¹, R²², R²³ and R²⁴ are Cl and/or F, wherein 1 or 2 of the residues R¹¹, R¹², R¹³, R¹⁴, R²¹, R²², R²³ and R²⁴ can be CN, and/or, and wherein 1 of the residues R¹¹, R¹², R¹³, R¹⁴, R²¹, R²², R²³ and R²⁴ can be H, and

R^{a} and R^{b} are independently from each other are H or an organic residue.

Takuro Teraoka, Satoru Hiroto and Hiroshi Shinokubo describe in Org. Lett. 2011, 13 (10), 2532-2535 the direct iridium-catalyzed tetraborylation of perylene diimides.

Glauco Battagliarin, Chen Li, Volker Enkelmann and Klaus Müllen describe in Org. Lett. 2011, 13 (12), pp. 3012-3015, the synthesis of 2,5,8,11-tetraboronate perylenediimide derivatives via a ruthenium catalyzed reaction and subsequent conversion to 2,5,8,11-tetra-iodo and tetra-amino perylenediimide derivatives.

Glauco Battagliarin, Yanfei Zhao, Chen Li and Klaus Müllen describe in Org. Lett. 2011, 13 (13), pp. 3399-3401, the synthesis and optical and electrochemical properties of 2,5,8,11-substituted perylenediimides

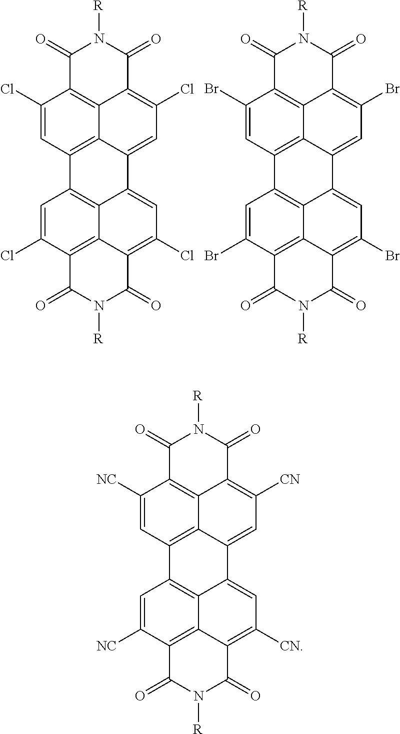

Unpublished European patent application 11156721.0 describes compounds of the formula

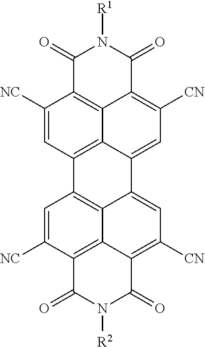

wherein

R¹ and R² are selected from H, C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl. Those compounds are prepared from a boron-containing precursor of the formula

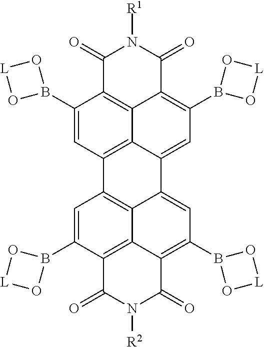

wherein R¹ and R² are as defined above and L is a linking group.

Unpublished European patent application 11165667.4 describes compounds of the formula

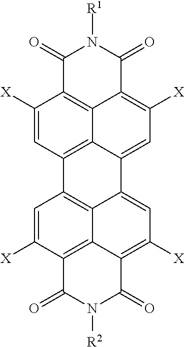

wherein X is Cl, Br or I and the use thereof as semiconducting material.

Unpublished European patent application 11177404.8 describes compounds of the formula

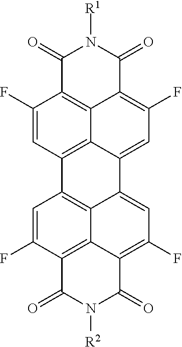

and the use thereof as semiconducting material.

WO 03/104232 describes 1,6,9,14-tetrasubstituted terrylene tetracarboxylic acid diimides of the formula

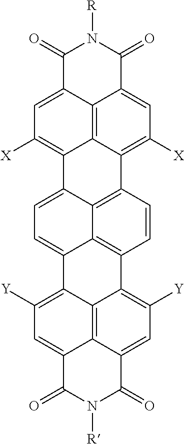

wherein

X and Y are each independently bromine; cyano;

aryloxy, arylthio, hetaryloxy or hetarylthio, each of which may be mono- or polysubstituted by C₁-C₁₂-alkyl, C₁-C₁₂-alkoxy, —COOR¹, —SO₃R¹, halogen, hydroxyl, carboxyl, cyano, —CONHR² and/or —NHCOR²;

a radical of the formula -L-R³; or a radical of the formula —NR²²;

R¹ is hydrogen or C₁-C₆-alkyl;

R² is hydrogen; C₁-C₁₈-alkyl; aryl or hetaryl, each of which may be substituted by C₁-C₆-alkyl, C₁-C₆-alkoxy, halogen, hydroxyl, carboxyl or cyano;

R³ is C₁-C₁₈-alkyl whose carbon chain may be interrupted by one or more —O—, —S—, —NR¹—, —CO— and/or —SO₂— groups, and may be mono- or polysubstituted by —COOR¹, —SO₃R¹, hydroxyl, cyano, C₁-C₆-alkoxy, aryl which may be substituted by C₁-C₁₈-alkyl or C₁-C₆-alkoxy, and/or a 5- to 7-membered heterocyclic radical which is bonded via a nitrogen atom and may contain further heteroatoms and be aromatic.

WO 2009/037283 describes a method for producing substrates coated with rylene tetracarboxylic acid diimides. The employed compounds do not comprise derivatives of terrylene diimides with electron withdrawing groups 2, 5, 10 and 13 position and derivatives of quaterrylene diimides with electron withdrawing groups in the 2, 5, 12 and 15 position.

US 2011/0079773 describes inter alia functionalized rylene imides of the general structure

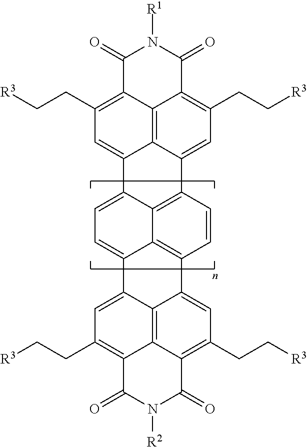

wherein n is 0, 1 or 2 and R¹, R² and R³ are more closely defined substituents, and their use in organic electronics.

It has now been found surprisingly, that derivatives of terrylene diimides with electron withdrawing groups in the 2, 5, 10 and 13 position and derivatives of quaterrylene diimides with electron withdrawing groups in the 2, 5, 12 and 15 position are particularly advantageous as semiconductor materials in organic electronics and organic photovoltaics.

### SUMMARY OF THE INVENTION

According to a first aspect of the present invention there is provided a compound of the general formula I

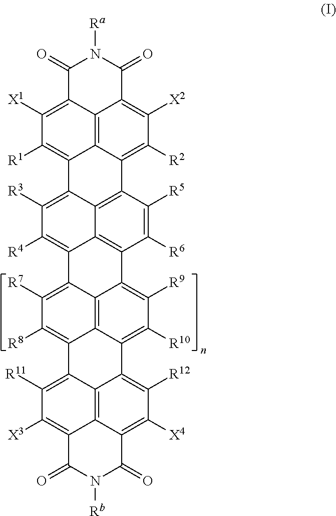

wherein
R^{a} and R^{b} are independently of one another selected from hydrogen and in each case unsubstituted or substituted alkyl, alkenyl, alkadienyl, alkynyl, cycloalkyl, bicycloalkyl, cycloalkenyl, heterocycloalkyl, aryl or heteroaryl,
X¹, X², X³ and X⁴ are independently of one another selected from F, Cl, Br, I, CN and B(OR^{c})₂,
   wherein R^{c} is selected from in each case unsubstituted or substituted alkyl, cycloalkyl or aryl, or wherein two radicals R^{c} may together form a divalent bridging group selected from in each case unsubstituted or substituted C₂-C₁₀-alkylene, C₃-C₆-cycloalkylene and C₆-C₁₄-arylene, wherein C₂-C₁₀-alkylene, C₃-C₆-cycloalkylene and C₆-C₁₄-arylene may carry one or more identical or different C₁-C₁₂-alkyl radicals,
R¹, R², R³, R⁴, R⁵, R⁶, R¹¹ and R¹² and, if present, R⁷, R⁸, R⁹ and R¹⁰ are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each sulfamoyl, alkylsulfonyl, arylsulfonyl, amidino, NE¹E², Where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino, (dihetaryl)amino.

According to a further aspect of the present invention there is provided a process for the preparation of a compound of the formula I.

According to a further aspect of the present invention there is provided an organic field effect transistor comprising a substrate having at least one gate structure, a source electrode and a drain electrode and at least one compound of the formula I as defined above and in the following as a semiconductor material.

The compounds of the formula (I) can be in principle used as n-semiconductors or as p-semiconductors. If a compound of the formula (I) acts as n-semiconductor or as p-semiconductors depends inter alia on the employed gate dielectric. Gate dielectrics are usually employed in the form of a self-assembled monolayer (SAM) of suitable compounds, e.g. silanes with more or less electronegative substituents, alkyl phosphonic acid, fluoroalkyl phosphonic acid, etc. By choosing a certain SAM gate dielectric or a certain mixture of different SAM gate dielectrics, it is possible to control the properties of the semiconductor material. In electronic devices that employ a combination of two different semiconductors, e.g. organic solar cells, it depends on the corresponding semiconductor material if a compound of the formula (I) acts as n-semiconductor or as p-semiconductor.

According to a further aspect of the present invention there is provided a substrate comprising a plurality of organic field-effect transistors, at least some of the field-effect transistors comprising at least one compound of the formula I as defined above and in the following.

According to a further aspect of the present invention there is provided a semiconductor unit comprising at least one substrate comprising a plurality of organic field-effect transistors, at least some of the field-effect transistors comprising at least one compound of the formula I as defined above and in the following.

According to a further aspect of the present invention there is provided an electroluminescent arrangement comprising an upper electrode, a lower electrode, wherein at least one of said electrodes is transparent, an electroluminescent layer and optionally an auxiliary layer, wherein the electroluminescent arrangement comprises at least one compound of the formula I as defined above and in the following.

In a preferred embodiment, the electroluminescent arrangement is in form of an organic light-emitting diode (OLED).

According to a further aspect of the present invention there is provided an organic solar cell comprising at least one compound of the formula (I) as defined above and in the following.

According to a further aspect of the present invention there is provided the use of a compound of the general formula I, as defined above and in the following, as a semiconductor material.

In a preferred embodiment, the compound of the general formula I are used as a semiconductor material in organic electronics or in organic photovoltaics.

### BRIEF DESCRIPTION OF THE DRAWINGS

FIG. 1 is a diagram showing the transfer characteristics of an OFET on the basis of the compound of example 3 prepared in accordance with the invention,

FIG. 2 is a diagram showing the output characteristics of an OFET on the basis of the compound of example 3.

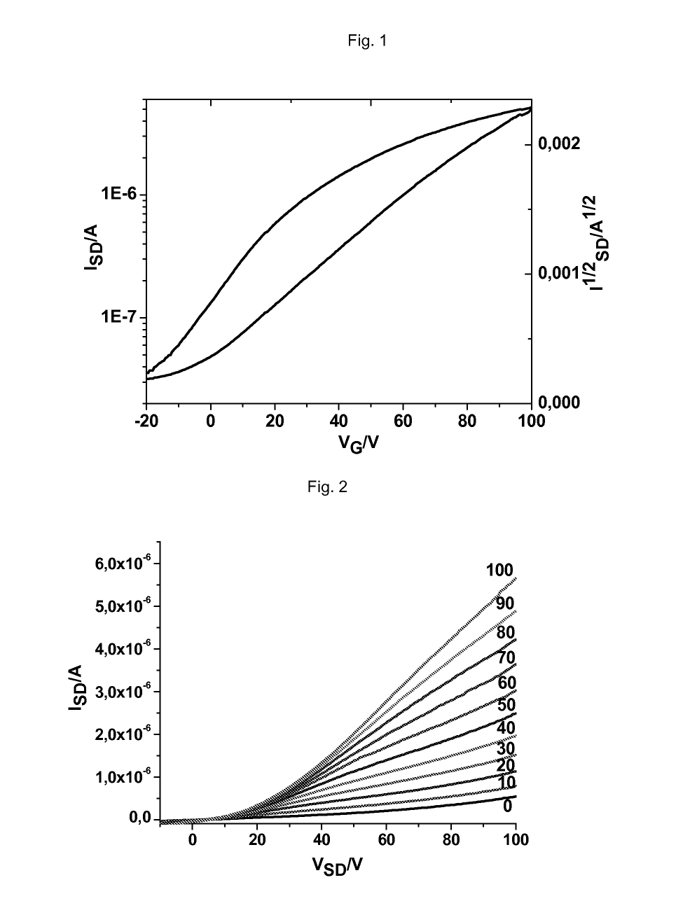

### DETAILED DESCRIPTION OF THE INVENTION

The expression “halogen” denotes in each case fluorine, bromine, chlorine or iodine, particularly chlorine, bromide or iodine.

As used herein, the expression “unsubstituted or substituted alkyl, alkenyl, alkadienyl, alkynyl, cycloalkyl, bicycloalkyl, cycloalkenyl, heterocycloalkyl, aryl or heteroaryl” refers to unsubstituted or substituted alkyl, unsubstituted or substituted alkenyl, unsubstituted or substituted alkadienyl, unsubstituted or substituted alkynyl, unsubstituted or substituted cycloalkyl, unsubstituted or substituted bicycloalkyl, unsubstituted or substituted cycloalkenyl, unsubstituted or substituted heterocycloalkyl, unsubstituted or substituted aryl or unsubstituted or substituted heteroaryl.

In the context of the invention, the expression “unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino” represents unsubstituted or substituted alkyl, unsubstituted or substituted alkoxy, unsubstituted or substituted alkylthio, unsubstituted or substituted (monoalkyl)amino, unsubstituted or substituted (dialkyl)amino, unsubstituted or substituted cycloalkyl, unsubstituted or substituted cycloalkoxy, unsubstituted or substituted cycloalkylthio, unsubstituted or substituted (monocycloalkyl)amino, unsubstituted or substituted (dicycloalkyl)amino, unsubstituted or substituted heterocycloalkyl, unsubstituted or substituted heterocycloalkoxy, unsubstituted or substituted heterocycloalkylthio, unsubstituted or substituted (monoheterocycloalkyl)amino, unsubstituted or substituted (diheterocycloalkyl)amino, unsubstituted or substituted aryl, unsubstituted or substituted aryloxy, unsubstituted or substituted arylthio, unsubstituted or substituted (monoaryl)amino, unsubstituted or substituted (diaryl)amino, unsubstituted or substituted hetaryl, unsubstituted or substituted hetaryloxy, unsubstituted or substituted hetarylthio, unsubstituted or substituted (monohetaryl)amino and unsubstituted or substituted (dihetaryl)amino.

In the context of the present invention, the expression “alkyl” comprises straight-chain or branched alkyl groups. Alkyl is preferably C₁-C₃₀-alkyl, more preferably C₁-C₂₀-alkyl and most preferably C₁-C₁₂-alkyl. Examples of alkyl groups are especially methyl, ethyl, n-propyl, isopropyl, n-butyl, isobutyl, sec-butyl, tert-butyl, n-pentyl, 1-methylbutyl, 1-ethylpropyl, neo-pentyl, n-hexyl, 1-methylpentyl, 2-methylpentyl, 1-ethylbutyl, 2-ethylbutyl, n-heptyl, 1-methylhexyl, 2-methylhexyl, 1-ethylpentyl, 1-propylbutyl, 2-ethylpentyl, n-octyl, 1-methylheptyl, 2-methylheptyl, 1-ethylhexyl, 2-ethylhexyl, 1-propylpentyl, 2-propylpentyl, n-nonyl, 1-methyloctyl, 2-methyloctyl, 1-ethylheptyl, 2-ethylheptyl, 1-propylhexyl, 2-propylhexyl, 1-butylpentyl, n-decyl, 2-methyldecyl, 1-methylnonyl, 2-methylnonyl, 1-ethyloctyl, 2-ethyloctyl, 1-propylheptyl, 2-propylheptyl, 1-butylhexyl, 2-butylhexyl, n-undecyl, 2-ethylnonyl, 1-propyloctyl, 2-propyloctyl, 1-butylheptyl, 2-butylheptyl, 1-pentylhexyl, n-dodecyl, 2-ethyldecyl, 2-propylnonyl, 1-butyloctyl, 2-butyloctyl, 1-pentylheptyl, 2-pentylheptyl, 2-propyldecyl, n-tridecyl, 1-pentyloctyl, 2-pentyloctyl, 1-hexylheptyl, 2-butylnonyl, n-tetradecyl, 1-hexyloctyl, 2-hexyloctyl, 2-pentylnonyl, 2-hexylnonyl, 2-pentyldecyl, 2-butyldecyl, n-hexadecyl, 1-heptyloctyl, 2-heptylnonyl, 2-hexyldecyl, 2-heptyldecyl, n-octadecyl, 2-octyldecyl, n-eicosyl, 2-nonylundecyl, 2-octylundecyl, 2-heptylundecyl, 2-hexylundecyl, 2-pentylundecyl, 2-butylundecyl, 2-propylundecyl, 2-ethylundecyl, 2-methylundecyl, 2-decyldodecyl, 2-nonyldodecyl, 2-octyldodecyl, 2-heptyldodecyl, 2-hexyldodecyl, 2-pentyldodecyl, 2-butyldodecyl, 2-propyldodecyl, 2-ethyldodecyl, 2-methyldodecyl, 2-undecyltridecyl, 2-decyltridecyl, 2-nonyltridecyl, 2-octyltridecyl, 2-heptyltridecyl, 2-hexyltridecyl, 2-pentyltridecyl, 2-butyltridecyl, 2-propyltridecyl, 2-ethyltridecyl, 2-methyltridecyl, 2-undecyltetradecyl, 2-decyltetradecyl, 2-nonyltetradecyl, 2-octyltetradecyl, 2-hetyltetradecyl, 2-hexyltetradecyl, 2-pentyltetradecyl, 2-butyltetradecyl, 2-propyltetradecyl, 2-ethyltetradecyl, 2-methyltetradecyl, 2-tetradecylhexadecyl, 2-tridecylhexadecyl, 2-dodecylhexadecyl, 2-undecylhexadecyl, 2-decylhexadecyl, 2-nonylhexadecyl, 2-octylhexadecyl, 2-heptylhexadecyl, 2-hexylhexadecyl, 2-pentylhexadecyl, 2-butylhexadecyl, 2-propylhexadecyl, 2-ethylhexadecyl, 2-methylhexadecyl, 2-dodecyloctadecyl, 2-undecyloctadecyl, 2-decyloctadecyl, 2-nonyloctadecyl, 2-octyloctadecyl, 2-heptyloctadecyl, 2-hexyloctadecyl, 2-pentyloctadecyl, 2-butyloctadecyl, 2-propyloctadecyl, 2-ethyloctadecyl, 2-methyloctadecyl, 2-decyleicosanyl, 2-nonyleicosanyl, 2-octyleicosanyl, 2-heptyleicosanyl, 2-hexyleicosanyl, 2-pentyleicosanyl, 2-butyleicosanyl, 2-propyleicosanyl, 2-ethyleicosanyl, 2-methyleicosanyl, 2-octadecyldocosanyl, 2-heptadecyldocosanyl, 2-hexadecyldocosanyl, 2-pentadecyldocosanyl, 2-tetradecyldocosanyl, 2-tridecyldocosanyl, 2-undecyldocosanyl, 2-decyldocosanyl, 2-nonyldocosanyl, 2-octyldocosanyl, 2-heptyldocosanyl, 2-hexyldocosanyl, 2-pentyldocosanyl, 2-butyldocosanyl, 2-propyldocosanyl, 2-ethyldocosanyl, 2-methyldocosanyl, 2-docosanyltetracosanyl, 2-hexadecyltetracosanyl, 2-pentadecyltetracosanyl, 2-pentadecyltetracosanyl, 2-tetradecyltetracosanyl, 2-tridecyltetracosanyl, 2-dodecyltetracosanyl, 2-undecyltetracosanyl, 2-decyltetracosanyl, 2-nonyltetracosanyl, 2-octyltetracosanyl, 2-heptyltetracosanyl, 2-hexyltetracosanyl, 2-pentyltetracosanyl, 2-butyltetracosanyl, 2-propyltetracosanyl, 2-ethyltetracosanyl, 2-methyltetracosanyl, 2-dodecyloctacosanyl, 2-undecyloctacosanyl, 2-decyloctacosanyl, 2-nonyloctacosanyl, 2-octyloctacosanyl, 2-heptyloctacosanyl, 2-hexyloctacosanyl, 2-pentyloctacosanyl, 2-butyloctacosanyl, 2-propyloctacosanyl, 2-ethyloctacosanyl and 2-methyloctacosanyl.

The expression alkyl also comprises alkyl radicals whose carbon chains may be interrupted by one or more nonadjacent groups which are selected from —O—, —S—, —NRⁿ—, —C(═O)—, —S(═O)— and/or —S(═O)₂—. Rⁿ is preferably hydrogen, alkyl, cycloalkyl, heterocycloalkyl, aryl or hetaryl.

Substituted alkyl groups may, depending on the length of the alkyl chain, have one or more (e.g. 1, 2, 3, 4, 5 or more than 5) substituents. These are preferably each independently selected from Si(alkyl)(O—Si(alkyl))₂, Si(aryl)(O—Si(alkyl))₂, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE¹E² where E¹ and E² are each independently hydrogen, alkyl, cycloalkyl, heterocycloalkyl, aryl or hetaryl. Cycloalkyl, heterocycloalkyl, aryl and hetaryl substituents of the alkyl groups may in turn be unsubstituted or substituted; suitable substituents are the substituents mentioned below for these groups.

Carboxylate and sulfonate respectively represent a derivative of a carboxylic acid function and a sulfonic acid function, especially a metal carboxylate or sulfonate, a carboxylic ester or sulfonic ester function or a carboxamide or sulfonamide function. Such derivatives include, for example, esters with C₁-C₄-alkanols, such as methanol, ethanol, n-propanol, isopropanol, n-butanol, sec-butanol and tert-butanol.

The above remarks regarding alkyl also apply to the alkyl moiety in alkoxy, alkylthio (=alkylsulfanyl), monoalkylamino and dialkylamino.

Alkylene represents a linear saturated hydrocarbon chain having from 1 to 10 and especially from 1 to 4 carbon atoms, such as ethane-1,2-diyl, propane-1,3-diyl, butane-1,4-diyl, pentane-1,5-diyl or hexane-1,6-diyl.

In the context of the present invention, the term “cycloalkyl” denotes a mono-, bi- or tricyclic hydrocarbon radical having usually from 3 to 20, preferably 3 to 12, more preferably 5 to 12, carbon atoms such as cyclopropyl, cyclobutyl, cyclopentyl, cyclohexyl, cycloheptyl, cyclooctyl, cyclododecyl, cyclopentadecyl, norbornyl, bicyclo[2.2.2]octyl or adamantyl.

Substituted cycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE³E⁴ where E³ and E⁴ are each independently hydrogen, alkyl, cycloalkyl, heterocycloalkyl, aryl or hetaryl. In the case of substitution, the cycloalkyl groups preferably bear one or more, for example one, two, three, four or five, C₁-C₆-alkyl groups. Examples of substituted cycloalkyl groups are especially 2- and 3-methylcyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec.-butylcyclohexyl, 2-, 3- and 4-tert-butylcyclohexyl, 2-, 3- and 4-methylcycloheptyl, 2-, 3- and 4-ethylcycloheptyl, 2-, 3- and 4-propylcycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methylcyclooctyl, 2-, 3-, 4- and 5-ethylcyclooctyl, 2-, 3-, 4- and 5-propylcyclooctyl.

The above remarks regarding cycloalkyl also apply to the cycloalkyl moiety in cycloalkoxy, cycloalkylthio (=cycloalkylsulfanyl), monocycloalkylamino and dicycloalkylamino.

In the context of the present invention, the term “aryl” refers to mono- or polycyclic aromatic hydrocarbon radicals. Aryl usually is an aromatic radical having 6 to 24 carbon atoms, preferably 6 to 20 carbon atoms, especially 6 to 14 carbon atoms as ring members. Aryl is preferably phenyl, naphthyl, indenyl, fluorenyl, anthracenyl, phenanthrenyl, naphthacenyl, chrysenyl, pyrenyl, coronenyl, perylenyl, etc., and more preferably phenyl or naphthyl.

Substituted aryls may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, O—Si(aryl)₃, Si(alkyl)(O—Si(alkyl)₃)₂, Si(aryl)(O—Si(alkyl)₃)₂, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. The alkyl, alkoxy, alkylamino, alkylthio, cycloalkyl, heterocycloalkyl, aryl and hetaryl substituents on the aryl may in turn be unsubstituted or substituted. Reference is made to the substituents mentioned above for these groups. The substituents on the aryl are preferably selected from alkyl, alkoxy, haloalkyl, haloalkoxy, aryl, fluorine, chlorine, bromine, cyano and nitro. Substituted aryl is more preferably substituted phenyl which generally bears 1, 2, 3, 4 or 5, preferably 1, 2 or 3, substituents.

Substituted aryl is preferably aryl substituted by at least one alkyl group (“alkaryl”, also referred to hereinafter as alkylaryl). Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl and 2-, 3- and 4-butoxyphenyl; 2-, 3- and 4-chlorophenyl, (2-chloro-6-methyl)phenyl, (2-chloro-6-ethyl)phenyl, (4-chloro-6-methyl)phenyl, (4-chloro-6-ethyl)phenyl.

The above remarks regarding aryl also apply to the aryl moiety in aryloxy, arylthio (=arylsulfanyl), monoarylamino and diarylamino.

In the context of the present invention, the expression “heterocycloalkyl” comprises nonaromatic, unsaturated or fully saturated, cycloaliphatic groups having generally 5 to 8 ring atoms, preferably 5 or 6 ring atoms. In the heterocycloalkyl groups, compared to the corresponding cycloalkyl groups, 1, 2, 3, 4 or more than 4 of the ring carbon atoms are replaced by heteroatoms or heteroatom-containing groups. The heteroatoms or heteroatom-containing groups are preferably selected from —O—, —S—, —NR^{e-}, —C(═O)—, —S(═O)— and/or —S(═O)₂—. R^{e} is preferably hydrogen, alkyl, cycloalkyl, heterocycloalkyl, aryl or hetaryl. Heterocycloalkyl is unsubstituted or optionally bears one or more, e.g. 1, 2, 3, 4, 5, 6 or 7, identical or different radicals. These are preferably each independently selected from alkyl, alkoxy, alkylamino, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. Examples of heterocycloalkyl groups are especially pyrrolidinyl, piperidinyl, 2,2,6,6-tetramethylpiperidinyl, imidazolidinyl, pyrazolidinyl, oxazolidinyl, morpholinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 2-oxazolinyl, 3-oxazolinyl, 4-oxazolinyl and dioxanyl.

Substituted heterocycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁷E⁸ where E⁷ and E⁸ are each independently hydrogen, alkyl, cycloalkyl, heterocycloalkyl, aryl or hetaryl. In the case of substitution, the heterocycloalkyl groups preferably bear one or more, for example one, two, three, four or five, C₁-C₆-alkyl groups.

The above remarks regarding heterocycloalkyl also apply to the heterocycloalkyl moiety in heterocycloalkoxy, heterocycloalkylthio (=heterocycloalkylsulfanyl), monoheterocycloalkylamino and diheterocycloalkylamino.

In the context of the present invention, the expression “hetaryl” (heteroaryl) comprises heteroaromatic, mono- or polycyclic groups. In addition to the ring carbon atoms, these have 1, 2, 3, 4 or more than 4 heteroatoms as ring members. The heteroatoms are preferably selected from oxygen, nitrogen, selenium and sulfur. The hetaryl groups have preferably 5 to 18, e.g. 5, 6, 8, 9, 10, 11, 12, 13 or 14, ring atoms.

Monocyclic hetaryl groups are preferably 5- or 6-membered hetaryl groups, such as 2-furyl (furan-2-yl), 3-furyl (furan-3-yl), 2-thienyl (thiophen-2-yl), 3-thienyl (thiophen-3-yl), selenophen-2-yl, selenophen-3-yl, 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Polycyclic hetaryl groups have 2, 3, 4 or more than 4 fused rings. The fused-on rings may be aromatic, saturated or partly unsaturated. Examples of polycyclic hetaryl groups are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl, benzothiadiazolyl, benzoxazinyl, benzopyrazolyl, benzimidazolyl, benzotriazolyl, benzotriazinyl, benzoselenophenyl, thienothiophenyl, thienopyrimidyl, thiazolothiazolyl, dibenzopyrrolyl (carbazolyl), dibenzofuranyl, dibenzothiophenyl, naphtho[2,3-b]thiophenyl, naphtha[2,3-b]furyl, dihydroindolyl, dihydroindolizinyl, dihydroisoindolyl, dihydroquinolinyl and dihydroisoquinolinyl.

Substituted hetaryl groups may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁹E¹⁰ where E⁹ and E¹⁰ are each independently hydrogen, alkyl, cycloalkyl, heterocycloalkyl, aryl or hetaryl. Halogen substituents are preferably fluorine, chlorine or bromine. The substituents are preferably selected from C₁-C₆-alkyl, C₁-C₆-alkoxy, hydroxyl, carboxyl, halogen and cyano.

The above remarks regarding hetaryl also apply to the hetaryl moiety in hetaryloxy, hetarylthio, monohetarylamino and dihetarylamino.

For the purposes of the present invention, the expression “acyl” refers to alkanoyl or aroyl groups which generally have from 2 to 11, preferably from 2 to 8, carbon atoms, for example the acetyl, propanoyl, butanoyl, pentanoyl, hexanoyl, heptanoyl-, 2-ethylhexanoyl, 2-propylheptanoyl, pivaloyl, benzoyl or naphthoyl group.

The groups NE¹E² are preferably N,N-dimethylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Fused ring systems can comprise alicyclic, aliphatic heterocyclic, aromatic and heteroaromatic rings and combinations thereof, hydroaromatic joined by fusion. Fused ring systems comprise two, three or more (e.g. 4, 5, 6, 7 or 8) rings. Depending on the way in which the rings in fused ring systems are joined, a distinction is made between ortho-fusion, i.e. each ring shares at least one edge or two atoms with each adjacent ring, and peri-fusion in which a carbon atom belongs to more than two rings. Preferred fused ring systems are ortho-fused ring systems.

Depending on the substitution pattern, the compounds of the formula I may have one or more centers of chirality, in which case they are present as mixtures of enantiomers or diastereomers. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

Specific examples of the radicals mentioned in the following formulae and their substituents are:

methyl, ethyl, propyl, isopropyl, butyl, isobutyl, tert-butyl, pentyl, isopentyl, neopentyl, tert-pentyl, hexyl, 2-methylpentyl, heptyl, 1-ethylpentyl, octyl, 2-ethylhexyl, isooctyl, nonyl, isononyl, decyl, isodecyl, undecyl, dodecyl, tridecyl, isotridecyl, tetradecyl, pentadecyl, hexadecyl, heptadecyl, octadecyl, nonadecyl and eicosyl (the above terms isooctyl, isononyl, isodecyl and isotridecyl are trivial terms and stem from the alcohols obtained by the oxo process);

2-methoxyethyl, 2-ethoxyethyl, 2-propoxyethyl, 2-isopropoxyethyl, 2-butoxyethyl, 2- and 3-methoxypropyl, 2- and 3-ethoxypropyl, 2- and 3-propoxypropyl, 2- and 3-butoxypropyl, 2- and 4-methoxybutyl, 2- and 4-ethoxybutyl, 2- and 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxamidecyl and 3,6,9,12-tetraoxatetradecyl;

2-methylthioethyl, 2-ethylthioethyl, 2-propylthioethyl, 2-isopropylthioethyl, 2-butylthioethyl, 2- and 3-methylthiopropyl, 2- and 3-ethylthiopropyl, 2- and 3-propylthiopropyl, 2- and 3-butylthiopropyl, 2- and 4-methylthiobutyl, 2- and 4-ethylthiobutyl, 2- and 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiamidecyl and 3,6,9,12-tetrathiatetradecyl;

2-monomethyl- and 2-monoethylaminoethyl, 2-dimethylaminoethyl, 2- and 3-dimethylaminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazamidecyl and 3,6,9,12-tetramethyl-3,6,9,12-tetraazamidecyl;

(1-ethylethylidene)aminoethylene, (1-ethylethylidene)aminopropylene, (1-ethylethylidene)aminobutylene, (1-ethylethylidene)aminodecylene and (1-ethylethylidene)aminododecylene; propan-2-on-1-yl, butan-3-on-1-yl, butan-3-on-2-yl and 2-ethylpentan-3-on-1-yl;

2-methylsulfoxidoethyl, 2-ethylsulfoxidoethyl, 2-propylsulfoxidoethyl, 2-isopropylsulfoxidoethyl, 2-butylsulfoxidoethyl, 2- and 3-methylsulfoxidopropyl, 2- and 3-ethylsulfoxidopropyl, 2- and 3-propylsulfoxidopropyl, 2- and 3-butylsulfoxidopropyl, 2- and 4-methylsulfoxidobutyl, 2- and 4-ethylsulfoxidobutyl, 2- and 4-propylsulfoxidobutyl and 4-butylsulfoxidobutyl;

2-methylsulfonylethyl, 2-ethylsulfonylethyl, 2-propylsulfonylethyl, 2-isopropylsulfonylethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylpropyl, 2- and 4-methylsulfonylbutyl, 2- and 4-ethylsulfonylbutyl, 2- and 4-propylsulfonylbutyl and 4-butylsulfonylbutyl;

carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxytetradecyl;

sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl;

2-hydroxyethyl, 2- and 3-hydroxypropyl, 1-hydroxyprop-2-yl, 3- and 4-hydroxybutyl, 1-hydroxybut-2-yl and 8-hydroxy-4-oxaoctyl;

2-cyanoethyl, 3-cyanopropyl, 3- and 4-cyanobutyl, 2-methyl-3-ethyl-3-cyanopropyl, 7-cyano-7-ethylheptyl and 4,7-dimethyl-7-cyanoheptyl;

2-chloroethyl, 2- and 3-chloropropyl, 2-, 3- and 4-chlorobutyl, 2-bromoethyl, 2- and 3-bromopropyl and 2-, 3- and 4-bromobutyl;

2-nitroethyl, 2- and 3-nitropropyl and 2-, 3- and 4-nitrobutyl;

methoxy, ethoxy, propoxy, isopropoxy, butoxy, isobutoxy, sec-butoxy, tert-butoxy, pentoxy, isopentoxy, neopentoxy, tert-pentoxy and hexoxy;

methylthio, ethylthio, propylthio, isopropylthio, butylthio, isobutylthio, sec-butylthio, tert-butylthio, pentylthio, isopentylthio, neopentylthio, tert-pentylthio and hexylthio;

ethynyl, 1- and 2-propynyl, 1-, 2- and 3-butynyl, 1-, 2-, 3- and 4-pentynyl, 1-, 2-, 3-, 4- and 5-hexynyl, 1-, 2-, 3-, 4-, 5-, 6-, 7-, 8- and 9-decynyl, 1-, 2-, 3-, 4-, 5-, 6-, 7-, 8-, 9-, 10- and 11-dodecynyl and 1-, 2-, 3-, 4-, 5-, 6-, 7-, 8-, 9-, 10-, 11-, 12-, 13-, 14-, 15-, 16- and 17-octadecynyl;

ethenyl, 1- and 2-propenyl, 1-, 2- and 3-butenyl, 1-, 2-, 3- and 4-pentenyl, 1-, 2-, 3-, 4- and 5-hexenyl, 1-, 2-, 3-, 4-, 5-, 6-, 7-, 8- and 9-decenyl, 1-, 2-, 3-, 4-, 5-, 6-, 7-, 8-, 9-, 10- and 11-dodecenyl and 1-, 2-, 3-, 4-, 5-, 6-, 7-, 8-, 9-, 10-, 11-, 12-, 13-, 14-, 15-, 16- and 17-octadecenyl;

methylamino, ethylamino, propylamino, isopropylamino, butylamino, isobutylamino, pentylamino, hexylamino, dimethylamino, methylethylamino, diethylamino, dipropylamino, diisopropylamino, dibutylamino, diisobutylamino, dipentylamino, dihexylamino, dicyclopentylamino, dicyclohexylamino, dicycloheptylamino, diphenylamino and dibenzylamino;

formylamino, acetylamino, propionylamino and benzoylamino;

carbamoyl, methylaminocarbonyl, ethylaminocarbonyl, propylaminocarbonyl, butylaminocarbonyl, pentylaminocarbonyl, hexylaminocarbonyl, heptylaminocarbonyl, octylaminocarbonyl, nonylaminocarbonyl, decylaminocarbonyl and phenylaminocarbonyl;

aminosulfonyl, N,N-dimethylaminosulfonyl, N,N-diethylaminosulfonyl, N-methyl-N-ethylaminosulfonyl, N-methyl-N-dodecylaminosulfonyl, N-dodecylaminosulfonyl, (N,N-dimethylamino)ethylaminosulfonyl, N,N-(propoxyethyl)dodecylaminosulfonyl, N,N-diphenylaminosulfonyl, N,N-(4-tert-butylphenyl)octadecylaminosulfonyl and N,N-bis(4-chlorophenyl)aminosulfonyl;

methoxycarbonyl, ethoxycarbonyl, propoxycarbonyl, isopropoxycarbonyl, hexoxycarbonyl, dodecyloxycarbonyl, octadecyloxycarbonyl, phenoxycarbonyl, (4-tert-butylphenoxy)carbonyl and (4-chlorophenoxy)carbonyl;

methoxysulfonyl, ethoxysulfonyl, propoxysulfonyl, isopropoxysulfonyl, butoxysulfonyl, isobutoxysulfonyl, tert-butoxysulfonyl, hexoxysulfonyl, dodecyloxysulfonyl, octadecyloxysulfonyl, phenoxysulfonyl, 1- and 2-naphthyloxysulfonyl, (4-tert_butylphenoxy)sulfonyl and (4-chlorophenoxy)sulfonyl;

diphenylphosphino, di(o-tolyl)phosphino and diphenylphosphinoxido;

fluorine, chlorine, bromine and iodine;

phenylazo, 2-naphthylazo, 2-pyridylazo and 2-pyrimidylazo;

cyclopropyl, cyclobutyl, cyclopentyl, 2- and 3-methylcyclopentyl, 2- and 3-ethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, cycloheptyl, 2-, 3- and 4-methylcycloheptyl, 2-, 3- and 4-ethylcycloheptyl, 3- and 4-propylcycloheptyl, 3- and 4-isopropylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl and 3-, 4- and 5-propylcyclooctyl; 3- and 4-hydroxycyclohexyl, 3- and 4-nitrocyclohexyl and 3- and 4-chlorocyclohexyl;

1-, 2- and 3-cyclopentenyl, 1-, 2-, 3- and 4-cyclohexenyl, 1-, 2- and 3-cycloheptenyl and 1-, 2-, 3- and 4-cyclooctenyl;

2-dioxanyl, 1-morpholinyl, 1-thiomorpholinyl, 2- and 3-tetrahydrofuryl, 1-, 2- and 3-pyrrolidinyl, 1-piperazyl, 1-diketopiperazyl and 1-, 2-, 3- and 4-piperidyl;

phenyl, 2-naphthyl, 2- and 3-pyrryl, 2-, 3- and 4-pyridyl, 2-, 4- and 5-pyrimidyl, 3-, 4- and 5-pyrazolyl, 2-, 4- and 5-imidazolyl, 2-, 4- and 5-thiazolyl, 3-(1,2,4-triazyl), 2-(1,3,5-triazyl), 6-quinaldyl, 3-, 5-, 6- and 8-quinolinyl, 2-benzoxazolyl, 2-benzothiazolyl, 5-benzothiadiazolyl, 2- and 5-benzimidazolyl and 1- and 5-isoquinolyl;

1-, 2-, 3-, 4-, 5-, 6- and 7-indolyl, 1-, 2-, 3-, 4-, 5-, 6- and 7-isoindolyl, 5-(4-methylisoindolyl), 5-(4-phenylisoindolyl), 1-, 2-, 4-, 6-, 7- and 8-(1,2,3,4-tetrahydroisoquinolinyl), 3-(5-phenyl)-(1,2,3,4-tetrahydroisoquinolinyl), 5-(3-dodecyl)-(1,2,3,4-tetrahydroisoquinolinyl), 1-, 2-, 3-, 4-, 5-, 6-, 7- and 8-(1,2,3,4-tetrahydroquinolinyl) and 2-, 3-, 4-, 5-, 6-, 7- and 8-chromanyl, 2-, 4- and 7-quinolinyl, 2-(4-phenylquinolinyl) and 2-(5-ethylquinolinyl);

2-, 3- and 4-methylphenyl, 2,4-, 3,5- and 2,6-dimethylphenyl, 2,4,6-trimethylphenyl, 2-, 3- and 4-ethylphenyl, 2,4-, 3,5- and 2,6-diethylphenyl, 2,4,6-triethylphenyl, 2-, 3- and 4-propylphenyl, 2,4-, 3,5- and 2,6-dipropylphenyl, 2,4,6-tripropylphenyl, 2-, 3- and 4-isopropylphenyl, 2,4-, 3,5- and 2,6-diisopropylphenyl, 2,4,6-triisopropylphenyl, 2-, 3- and 4-butylphenyl, 2,4-, 3,5- and 2,6-dibutylphenyl, 2,4,6-tributylphenyl, 2-, 3- and 4-isobutylphenyl, 2,4-, 3,5- and 2,6-diisobutylphenyl, 2,4,6-triisobutylphenyl, 2-, 3- and 4-sec-butylphenyl, 2,4-, 3,5- and 2,6-di-sec-butylphenyl and 2,4,6-tri-sec-butylphenyl;

2-, 3- and 4-methoxyphenyl, 2,4-, 3,5- and 2,6-dimethoxyphenyl, 2,4,6-trimethoxyphenyl, 2-, 3- and 4-ethoxyphenyl, 2,4-, 3,5- and 2,6-diethoxyphenyl, 2,4,6-triethoxyphenyl, 2-, 3- and 4-propoxyphenyl, 2,4-, 3,5- and 2,6-dipropoxyphenyl, 2-, 3- and 4-isopropoxyphenyl, 2,4- and 2,6-diisopropoxyphenyl and 2-, 3- and 4-butoxyphenyl; 2-, 3- and 4-chlorophenyl and 2,4-, 3,5- and 2,6-dichlorophenyl; 2-, 3- and 4-hydroxyphenyl and 2,4-, 3,5- and 2,6-dihydroxyphenyl; 2-, 3- and 4-cyanophenyl; 3- and 4-carboxyphenyl; 3- and 4-carboxamidophenyl, 3- and 4-N-methylcarboxamidophenyl and 3- and 4-N-ethylcarboxamidophenyl; 3- and 4-acetylaminophenyl, 3- and 4-propionylaminophenyl and 3- and 4-butyrylaminophenyl; 3- and 4-N-phenylaminophenyl, 3- and 4-N-(o-tolyl)aminophenyl, 3- and 4-N-(m-tolyl)aminophenyl and 3- and 4-(p-tolyl)aminophenyl; 3- and 4-(2-pyridyl)aminophenyl, 3- and 4-(3-pyridyl)aminophenyl, 3- and 4-(4-pyridyl)aminophenyl, 3- and 4-(2-pyrimidyl)aminophenyl and 4-(4-pyrimidyl)aminophenyl;

4-phenylazophenyl, 4-(1-naphthylazo)phenyl, 4-(2-naphthylazo)phenyl, 4-(4-naphthylazo)phenyl, 4-(2-pyridylazo)phenyl, 4-(3-pyridylazo)phenyl, 4-(4-pyridylazo)phenyl, 4-(2-pyrimidylazo)phenyl, 4-(4-pyrimidylazo)phenyl and 4-(5-pyrimidylazo)phenyl;

phenoxy, phenylthio, 2-naphthoxy, 2-naphthylthio, 2-, 3- and 4-pyridyloxy, 2-, 3- and 4-pyridylthio, 2-, 4- and 5-pyrimidyloxy and 2-, 4- and 5-pyrimidylthio.

Specific examples of radicals containing fluorine are the following:

2,2,2-trifluoroethyl, 2,2,3,3,3-pentafluoropropyl, 2,2-difluoroethyl, 2,2,2-trifluoro-1-phenylethylamin, 1-benzyl-2,2,2-trifluoroethyl, 2-bromo-2,2-difluoroethyl, 2,2,2-trifluoro-1-pyridin-2-ylethyl, 2,2-difluoropropyl, 2,2,2-trifluoro-1-(4-methoxyphenyl)ethylamin, 2,2,2-trifluoro-1-phenylethylamin, 2,2-difluoro-1-phenylethylamin, 1-(4-bromo-phenyl)-2,2,2-trifluoroethyl, 3-bromo-3,3-difluoropropyl, 3,3,3-trifluoropropylamin, 3,3,3-trifluoro-n-propyl, 1H,1H,2H,2H-perfluorodecyl, 3-(perfluorooctyl)propyl, pentafluorophenyl, 2,3,5,6-tetrafluorophenyl, 4-cyano-(2,3,5,6)-tetrafluorophenyl, 4-carboxy-2,3,5,6-tetrafluorophenyl, 2,4-difluorophenyl, 2,4,5-trifluorophenyl, 2,4,6-trifluorophenyl, 2,5-difluorophenyl, 2-fluoro-5-nitrophenyl, 2-fluoro-5-trifluoromethylphenyl, 2-fluoro-5-methylphenyl, 2,6-difluorophenyl, 4-carboxamido-2,3,5,6-tetrafluorophenyl, 2-bromo-4,6-difluorophenyl, 4-bromo-2-fluorophenyl, 2,3-difluorophenyl, 4-chloro-2-fluorophenyl, 2,3,4-trifluorophenyl, 2-fluoro-4-iodphenyl, 4-bromo-2,3,5,6-tetrafluorophenyl, 2,3,6-trifluorophenyl, 2-bromo-3,4,6-trifluorophenyl, 2-bromo-4,5,6-trifluorophenyl, 4-bromo-2,6-difluorophenyl, 2,3,4,5-tetrafluorophenyl, 2,4-difluoro-6-nitrophenyl, 2-fluoro-4-nitrophenyl, 2-chloro-6-fluorophenyl, 2-fluoro-4-methylphenyl, 3-chloro-2,4-difluorophenyl, 2,4-dibromo-6-fluorophenyl, 3,5-dichloro-2,4-difluorophenyl, 4-cyano-1-fluorophenyl, 1-chloro-4-fluorophenyl, 2-fluoro-3-trifluoromethylphenyl, 2-trifluoromethyl-6-fluorophenyl, 2,3,4,6-tetrafluorophenyl, 3-chloro-2-fluorophenyl, 5-chloro-2-fluorophenyl, 2-bromo-4-chloro-6-fluorophenyl, 2,3-dicyano-4,5,6-trifluorophenyl, 2,4,5-trifluoro-3-carboxyphenyl, 2,3,4-trifluoro-6-carboxyphenyl, 2,3,5-trifluorophenyl, 4-trifluoromethyl-1,2,3,5,6-tetrafluorophenyl, 1-fluoro-5-carboxyphenyl, 2-chloro-4,6-difluorophenyl, 6-bromo-3-chloro-2,4-difluorophenyl, 2,3,4-trifluoro-6-nitrophenyl, 2,5-difluoro-4-cyanophenyl, 2,5-difluoro-4-trifluoromethylphenyl, 2,3-difluoro-6-nitrophenyl, 4-trifluoromethyl-2,3-difluorophenyl, 2-bromo-4,6-difluorophenyl, 4-bromo-2-fluorophenyl, 2-nitrotetrafluorophenyl, 2,2′,3,3′,4′,5,5′,6,6′-nonabiphenyl, 2-nitro-3,5,6-trifluorophenyl, 2-bromo-6-fluorophenyl, 4-chloro-2-fluoro-6-iodphenyl, 2-fluoro-6-carboxyphenyl, 2,4-difluoro-3-trifluorophenyl, 2-fluoro-4-trifluorophenyl, 2-fluoro-4-carboxyphenyl, 4-bromo-2,5-difluorophenyl, 2,5-dibromo-3,4,6-trifluorophenyl, 2-fluoro-5-methylsulphonylpenyl, 5-bromo-2-fluorophenyl, 2-fluoro-4-hydroxymethylphenyl, 3-fluoro-4-bromomethylphenyl, 2-nitro-4-trifluoromethylphenyl, 4-trifluoromethylphenyl, 2-bromo-4-trifluoromethylphenyl, 2-bromo-6-chloro-4-(trifluoromethyl)phenyl, 2-chloro-4-trifluoromethylphenyl, 3-nitro-4-(trifluoromethyl)phenyl, 2,6-dichloro-4-(trifluoromethyl)phenyl, 4-trifluorophenyl, 2,6-dibromo-4-(trifluoromethyl)phenyl, 4-trifluoromethyl-2,3,5,6-tetrafluorophenyl, 3-fluoro-4-trifluoromethylphenyl, 2,5-difluoro-4-trifluoromethylphenyl, 3,5-difluoro-4-trifluoromethylphenyl, 2,3-difluoro-4-trifluoromethylphenyl, 2,4-bis(trifluoromethyl)phenyl, 3-chloro-4-trifluoromethylphenyl, 2-bromo-4,5-di(trifluoromethyl)phenyl, 5-chloro-2-nitro-4-(trifluoromethyl)phenyl, 2,4,6-tris(trifluoromethyl)phenyl, 3,4-bis(trifluoromethyl)phenyl, 2-fluoro-3-trifluoromethylphenyl, 2-Iod-4-trifluoromethylphenyl, 2-nitro-4,5-bis(trifluoromethyl)phenyl, 2-methyl-4-(trifluoromethyl)phenyl, 3,5-dichloro-4-(trifluoromethyl)phenyl, 2,3,6-trichloro-4-(trifluoromethyl)phenyl, 4-(trifluoromethyl)benzyl, 2-fluoro-4-(trifluoromethyl)benzyl, 3-fluoro-4-(trifluoromethyl)benzyl, 3-chloro-4-(trifluoromethyl)benzyl, 4-fluorophenethyl, 3-(trifluoromethyl)phenethyl, 2-chloro-6-fluorophenethyl, 2,6-dichlorophenethyl, 3-fluorophenethyl, 2-fluorophenethyl, (2-trifluoromethyl)phenethyl, 4-fluorophenethyl, 3-fluorophenethyl, 4-trifluoromethylphenethyl, 2,3-difluorophenethyl, 3,4-difluorophenethyl, 2,4-difluorophenethyl, 2,5-difluorophenethyl, 3,5-difluorophenethyl, 2,6-difluorophenethyl, 4-(4-fluorophenyl)phenethyl, 3,5-di(trifluoromethyl)phenethyl, pentafluorophenethyl, 2,4-di(trifluoromethyl)phenethyl, 2-nitro-4-(trifluoromethyl)phenethyl, (2-fluoro-3-trifluoromethyl)phenethyl, (2-fluoro-5-trifluoromethyl)phenethyl, (3-fluoro-5-trifluoromethyl)phenethyl, (4-fluoro-2-trifluoromethyl)phenethyl, (4-fluoro-3-trifluoromethyl)phenethyl, (2-fluoro-6-trifluoromethyl)phenethyl, (2,3,6-trifluoro)phenethyl, (2,4,5-trifluoro)phenethyl, (2,4,6-trifluoro)phenethyl, (2,3,4-trifluoro)phenethyl, (3,4,5-trifluoro)phenethyl, (2,3,5-trifluoro)phenethyl, (2-chloro-5-fluoro)phenethyl, (3-fluoro-4-trifluoromethyl)phenethyl, (2-chloro-5-trifluoromethyl)phenethyl, (2-fluoro-3-chloro-5-trifluoromethyl)phenethyl, (2-fluoro-3-chloro)phenethyl, (4-fluoro-3-chloro)phenethyl, (2-fluoro-4-chloro)phenethyl, (2,3-difluoro-4-methyl)phenethyl-, 2,6-difluoro-3-chlorophenethyl, (2,6-difluoro-3-methyl)phenethyl, (2-trifluoromethyl-5-chloro)phenethyl, (6-chloro-2-fluoro-5-methyl)phenethyl, (2,4-dichloro-5-fluoro)phenethyl, 5-chloro-2-fluorophenethyl, (2,5-difluoro-6-chloro)phenethyl, (2,3,4,5-tetrafluoro)phenethyl, (2-fluoro-4-trifluoromethyl)phenethyl, 2,3-(difluoro-4-trifluoromethyl)phenethyl, (2,5-di(trifluoromethyl))phenethyl, 2-fluoro-3,5-dibromophenethyl, (3-fluoro-4-nitro)phenethyl, (2-bromo-4-trifluoromethyl)phenethyl, 2-(bromo-5-fluoro)phenethyl, (2,6-difluoro-4-bromo)phenethyl, (2,6-difluoro-4-chloro)phenethyl, (3-chloro-5-fluoro)phenethyl, (2-bromo-5-trifluoromethyl)phenethyl and the like.

In the compounds of the formula (I), the R^{a} and R^{b} radicals may have identical or different definitions. In a preferred embodiment, the R^{a} and R^{b} radicals have identical definitions.

In a preferred embodiment, at least one of the radicals R^{a} and R^{b} is hydrogen. In particular, R^{a} and R^{b} have the same meaning and are both hydrogen.

In a preferred embodiment, at least one of the radicals R^{a} and R^{b} is a linear C₁-C₃₀-alkyl radical. Preferred linear alkyl groups are methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl. In particular, R^{a} and R^{b} have the same meaning and are both a linear C₁-C₃₀-alkyl radical.

In a preferred embodiment, at least one of the radicals R^{a} and R^{b} is a branched C₃-C₃₀-alkyl radical. In particular, R^{a} and R^{b} have the same meaning and are both a branched C₃-C₃₀-alkyl radical.

Preferably at least one of the radicals R^{a} and R^{b} is selected from radicals of the general formula (II)

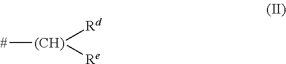

in which
# is a bonding site, and
R^{d} and R^{e} are independently selected from C₁- to C₂₈-alkyl, where the sum of the carbon atoms of the R^{d} and R^{e} radicals is an integer from 2 to 29.

In a preferred embodiment of the compounds (I), the radicals R^{a} and R^{b} are independently selected from radicals of the general formula (II). In particular, R^{a} and R^{b} have the same meaning and are selected from radicals of the general formula (II).

Preferably, in the formula (II), the R^{d} and R^{e} radicals are selected from C₁- to C₁₂-alkyl, especially C₁- to C₈-alkyl. In certain embodiments, the radical of the general formula (II) has at least one asymmetric carbon atoms. One center of chirality is the carbon atom carrying radicals R^{d} and R^{e}, wherein R^{d} is different from R^{e}. Examples are (R)-1-methylpropyl, (S)-1-methylpropyl, (R)-1-methylbutyl, (S)-1-methylbutyl, (R)-1-methylpentyl, (S)-1-methylpentyl, (R)-1-methylhexyl, (S)-1-methylhexyl, (R)-1-ethylbutyl and (S)-1-ethylbutyl, in particular (R)-1-methylpentyl and (S)-1-methylpentyl.

Preferred radicals of the formula (II) are:

1-ethylpropyl, 1-methylpropyl, 1-propylbutyl, 1-ethylbutyl, 1-methylbutyl, 1-butylpentyl, 1-propylpentyl, 1-ethylpentyl, 1-methylpentyl, 1-pentylhexyl, 1-butylhexyl, 1-propylhexyl, 1-ethylhexyl, 1-methylhexyl, 1-hexylheptyl, 1-pentylheptyl, 1-butylheptyl, 1-propylheptyl, 1-ethylheptyl, 1-methylheptyl, 1-heptyloctyl, 1-hexyloctyl, 1-pentyloctyl, 1-butyloctyl, 1-propyloctyl, 1-ethyloctyl, 1-methyloctyl, 1-octylnonyl, 1-heptylnonyl, 1-hexylnonyl, 1-pentylnonyl, 1-butylnonyl, 1-propylnonyl, 1-ethylnonyl, 1-methylnonyl, 1-nonyldecyl, 1-octyldecyl, 1-heptyldecyl, 1-hexyldecyl, 1-pentyldecyl, 1-butyldecyl, 1-propyldecyl, 1-ethyldecyl, 1-methyldecyl, 1-decylundecyl, 1-nonylundecyl, 1-octylundecyl, 1-heptylundecyl, 1-hexylundecyl, 1-pentylundecyl, 1-butylundecyl, 1-propylundecyl, 1-ethylundecyl, 1-methylundecyl, 1-undecyldodecyl, 1-decyldodecyl, 1-nonyldodecyl, 1-octyldodecyl, 1-heptyldodecyl, 1-hexyldodecyl, 1-pentyldodecyl, 1-butyldodecyl, 1-propyldodecyl, 1-ethyldodecyl, 1-methyldodecyl, 1-dodecyltridecyl, 1-undecyltridecyl, 1-decyltridecyl, 1-nonyltridecyl, 1-octyltridecyl, 1-heptyltridecyl, 1-hexyltridecyl, 1-pentyltridecyl, 1-butyltridecyl, 1-propyltridecyl, 1-ethyltridecyl, 1-methyltridecyl, 1-tridecyltetradecyl, 1-undecyltetradecyl, 1-decyltetradecyl, 1-nonyltetradecyl, 1-octyltetradecyl, 1-heptyltetradecyl, 1-hexyltetradecyl, 1-pentyltetradecyl, 1-butyltetradecyl, 1-propyltetradecyl, 1-ethyltetradecyl, 1-methyltetradecyl, 1-pentadecylhexadecyl, 1-tetradecylhexadecyl, 1-tridecylhexadecyl, 1-dodecylhexadecyl, 1-undecylhexadecyl, 1-decylhexadecyl, 1-nonylhexadecyl, 1-octylhexadecyl, 1-heptylhexadecyl, 1-hexylhexadecyl, 1-pentylhexadecyl, 1-butylhexadecyl, 1-propylhexadecyl, 1-ethylhexadecyl, 1-methylhexadecyl, 1-hexadecyloctadecyl, 1-pentadecyloctadecyl, 1-tetradecyloctadecyl, 1-tridecyloctadecyl, 1-dodecyloctadecyl, 1-undecyloctadecyl, 1-decyloctadecyl, 1-nonyloctadecyl, 1-octyloctadecyl, 1-heptyloctadecyl, 1-hexyloctadecyl, 1-pentyloctadecyl, 1-butyloctadecyl, 1-propyloctadecyl, 1-ethyloctadecyl, 1-methyloctadecyl, 1-nonadecyleicosanyl, 1-octadecyleicosanyl, 1-heptadecyleicosanyl, 1-hexadecyleicosanyl, 1-pentadecyleicosanyl, 1-tetradecyleicosanyl, 1-tridecyleicosanyl, 1-dodecyleicosanyl, 1-undecyleicosanyl, 1-decyleicosanyl, 1-nonyleicosanyl, 1-octyleicosanyl, 1-heptyleicosanyl, 1-hexyleicosanyl, 1-pentyleicosanyl, 1-butyleicosanyl, 1-propyleicosanyl, 1-ethyleicosanyl, 1-methyleicosanyl, 1-eicosanyldocosanyl, 1-nonadecyldocosanyl, 1-octadecyldocosanyl, 1-heptadecyldocosanyl, 1-hexadecyldocosanyl, 1-pentadecyldocosanyl, 1-tetradecyldocosanyl, 1-tridecyldocosanyl, 1-undecyldocosanyl, 1-decyldocosanyl, 1-nonyldocosanyl, 1-octyldocosanyl, 1-heptyldocosanyl, 1-hexyldocosanyl, 1-pentyldocosanyl, 1-butyldocosanyl, 1-propyldocosanyl, 1-ethyldocosanyl, 1-methyldocosanyl, 1-tricosanyltetracosanyl, 1-docosanyltetracosanyl, 1-nonadecyltetracosanyl, 1-octadecyltetracosanyl, 1-heptadecyltetracosanyl, 1-hexadecyltetracosanyl, 1-pentadecyltetracosanyl, 1-pentadecyltetracosanyl, 1-tetradecyltetracosanyl, 1-tridecyltetracosanyl, 1-dodecyltetracosanyl, 1-undecyltetracosanyl, 1-decyltetracosanyl, 1-nonyltetracosanyl, 1-octyltetracosanyl, 1-heptyltetracosanyl, 1-hexyltetracosanyl, 1-pentyltetracosanyl, 1-butyltetracosanyl, 1-propyltetracosanyl, 1-ethyltetracosanyl, 1-methyltetracosanyl, 1-heptacosanyloctacosanyl, 1-hexacosanyloctacosanyl, 1-pentacosanyloctacosanyl, 1-tetracosanyloctacosanyl, 1-tricosanyloctacosanyl, 1-docosanyloctacosanyl, 1-nonadecyloctacosanyl, 1-octadecyloctacosanyl, 1-heptadecyloctacosanyl, 1-hexadecyloctacosanyl, 1-hexadecyloctacosanyl, 1-pentadecyloctacosanyl, 1-tetradecyloctacosanyl, 1-tridecyloctacosanyl, 1-dodecyloctacosanyl, 1-undecyloctacosanyl, 1-decyloctacosanyl, 1-nonyloctacosanyl, 1-octyloctacosanyl, 1-heptyloctacosanyl, 1-hexyloctacosanyl, 1-pentyloctacosanyl, 1-butyloctacosanyl, 1-propyloctacosanyl, 1-ethyloctacosanyl, 1-methyloctacosanyl.

Particularly preferred radicals of the formula (II) are:

1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1-propylheptyl, 1-propyloctyl, 1-butylpentyl, 1-butylhexyl, 1-butylheptyl, 1-butyloctyl, 1-pentylhexyl, 1-pentylheptyl, 1-pentyloctyl, 1-hexylheptyl, 1-hexyloctyl, 1-heptyloctyl.

Preferably, at least one of the radicals R^{a} and R^{b} is selected from perfluoro-C₁-C₃₀-alkyl, 1H, 1H-perfluoro-C₂-C₃₀-alkyl or 1H, 1H,2H,2H-perfluoro-C₃-C₃₀-alkyl.

Preferably, in the compounds of the formula (I) R^{a} and R^{b} are each independently selected from perfluoro-C₁-C₃₀-alkyl. More preferably, R¹ and R² have the same meaning and are both perfluoro-C₁-C₃₀-alkyl.

Additionally preferably, in the compounds of the formula (I) R^{a} and R^{b} are each independently selected from 1H,1H-perfluoro-C₂-C₃₀-alkyl. More preferably, R¹ and R² have the same meaning and are both 1H,1H-perfluoro-C₂-C₃₀-alkyl.

Additionally preferably, in the compounds of the formula (I) R^{a} and R^{b} are each independently selected from 1H,1H,2H,2H-perfluoro-C₃-C₃₀-alkyl. More preferably, R¹ and R² have the same meaning and are 1H,1H,2H,2H-perfluoro-C₃-C₃₀-alkyl.

In a preferred embodiment, the radicals R^{a} and R^{b} are each independently perfluoro-C₁-C₂₀-alkyl or 1H,1H-perfluoro-C₂-C₂₀-alkyl or 1H,1H,2H,2H-perfluoro-C₃-C₂₀-alkyl.

In particular, the radicals R^{a} and R^{b} are each independently perfluoro-C₁-C₁₀-alkyl or 1H,1H-perfluoro-C₂-C₁₀-alkyl or 1H,1H,2H,2H-perfluoro-C₃-C₁₀-alkyl.

In a preferred embodiment, at least one of the radicals R^{a} and R^{b} is selected from CF₃, C₂F₅, n-C₃F₇, n-C₄F₉, n-C₅F₁₁, n-C₆F₁₃, CF(CF₃)₂, C(CF₃)₃, CF₂CF(CF₃)₂, CF(CF₃)(C₂F₅), CH₂—CF₃, CH₂—C₂F₅, CH₂-(n-C₃F₇), CH₂-(n-C₄F₉), CH₂-(n-C₅F₁₁), CH₂—(n-C₆F₁₃), CH₂—CF(CF₃)₂, CH₂—C(CF₃)₃, CH₂—CF₂CF(CF₃)₂, CH₂—CF(CF₃)(C₂F₅), CH₂—CH₂—CF₃, CH₂—CH₂—C₂F₅, CH₂—CH₂-(n-C₃F₇), CH₂—CH₂-(n-C₄F₉), CH₂—CH₂-(n-C₅F₁₁), CH₂—CH₂-(n-C₆F₁₃), CH₂—CH₂—CF(CF₃)₂, CH₂—CH₂—C(CF₃)₃, CH₂—CH₂—CF₂CF(CF₃)₂ and CH₂—CH₂—CF(CF₃)(C₂F₅).

In a special embodiment, the afore-mentioned fluorinated radicals R^{a} and R^{b} have the same meaning.

R^{a} and R^{b} are preferably both CH₂—CF₃, CH₂—C₂F₅ or CH₂-(n-C₃F₇).

In a preferred embodiment, at least one of the radicals R^{a} and R^{b} is selected from C₁-C₁₂-alkyl which carries one radical selected from Si(C₁-C₈-alkyl)(O—Si(C₁-C₈-alkyl)₃)₂, Si(phenyl)(O—Si(C₁-C₈-alkyl)₃)₂ and phenyl. More preferably, at least one of the radicals R^{a} and R^{b} is selected from C₃-C₁₂-alkyl, in particular linear C₃-C₁₂-alkyl, which carries one radical selected from Si(C₁-C₈-alkyl)(O—Si(C₁-C₈-alkyl)₃)₂, Si(phenyl)(O—Si(C₁-C₈-alkyl)₃)₂ and phenyl. In particular, at least one of the radicals R^{a} and R^{b} is selected from C₃-C₁₂-alkyl which carries one radical selected from Si(C₁-C₄-alkyl)(O—Si(C₁-C₄-alkyl)₃)₂, Si(phenyl)(O—Si(C₁-C₄-alkyl)₃)₂ and phenyl.

In a preferred embodiment, at least one of the radicals R^{a} and R^{b} is selected from [Si(C₁-C₈-alkyl)(O—Si(C₁-C₈-alkyl)₃)₂]-phenylene and (C₆H₅)₃Si—O—.

In a preferred embodiment, at least one of the radicals R^{a} and R^{b} is selected from

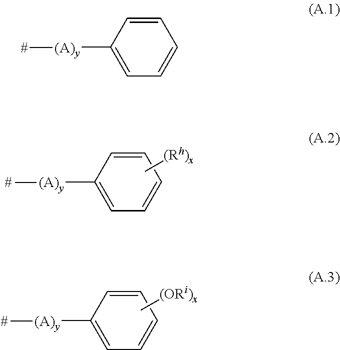

wherein
# represents the bonding side to a nitrogen atom,
A where present, is a C₁-C₁₀-alkylene group which may be interrupted by one or more nonadjacent groups which are selected from —O— and —S—,
y is 0 or 1,
R^{h} is selected independently of one another from C₁-C₃₀-alkyl, C₁-C₃₀-fluoroalkyl, fluorine, chlorine, bromine, NE¹E², nitro and cyano, where E¹ und E², independently of one another, are hydrogen, alkyl, cycloalkyl, heterocycloalkyl, aryl or hetaryl,
Rⁱ is selected independently of one another from C₁-C₃₀-alkyl,
x in formulae A.2 and A.3 is 1, 2, 3, 4 or 5.

In a preferred embodiment of the compounds (I), the radicals R^{a} and R^{b} are independently selected from radicals of the general formulae (A.1), (A.2) and (A.3). In particular, R¹ and R² have the same meaning and are selected from radicals of the general formulae (A.1), (A.2) and (A.3).

Preferably, in the radical of the formula A.2, the R^{h} radicals are selected from C₁-C₁₂-alkyl or C₁-C₁₂-fluoroalkyl. In particular, in the radical of the formula A.2, the R^{h} radicals are selected from C₁-C₄-alkyl and C₁-C₄-fluoroalkyl.

Preferably, in the radical of the formula A.3, the Rⁱ radicals are selected from C₁-C₁₂-alkyl.

In a preferred embodiment, in the compounds of the formula (I) R^{a} and R^{b} are each independently selected from radicals of the formula A.2. Preferably, R^{a} and R^{b} are each independently selected from phenyl-(C₁-C₃₀)-alkyl groups, wherein the benzene ring of the phenylalkyl group bears 1, 2, 3, 4 or 5 substituents, independently selected from F, Cl, Br, CN, C₁-C₃₀-alkyl and perfluoro-C₁-C₃₀-alkyl and the phenylalkyl group is attached to the imide nitrogen atom via the alkyl moiety of the phenylalkyl group.

More preferably, R^{a} and R^{b} have the same meaning and are selected from phenyl-(C₁-C₃₀)-alkyl groups, wherein the benzene ring of the phenylalkyl group bears 1, 2, 3, 4 or 5 substituents, independently selected from F, Cl, Br, CN, C₁-C₃₀-alkyl and perfluoro-C₁-C₃₀-alkyl. In particular, R¹ and R² have the same meaning and are selected from phenyl-(C₁-C₄)-alkyl groups, wherein the benzene ring of the phenylalkyl group bears 1, 2, 3, 4 or 5 substituents, independently selected from F, Cl, Br, CN, C₁-C₃₀-alkyl and perfluoro-C₁-C₁₂-alkyl.

Examples of preferred radicals of the formula A.1 are mentioned in the following table 1. In a preferred embodiment, in the compounds of the formula (I) R¹ and R² are each independently selected from radicals of the formula A.1 mentioned in the following table 1. In particular, R^{a} and R^{b} have the same meaning and are selected from radicals of the formula A.1 mentioned in the following table 1.

TABLE 1
(preferred radicals of the formula A.1):

| | | |
|---|---|---|
| 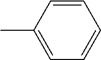 | 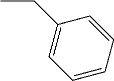 | 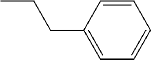 |

Examples of preferred radicals of the formula A.2 are mentioned in the following table 2. In a preferred embodiment, in the compounds of the formula (I) R^{a} and R^{b} are each independently selected from radicals of the formula A.2 mentioned in the following table 2. In particular, R^{a} and R^{b} have the same meaning and are selected from radicals of the formula A.2 mentioned in the following table 2.

TABLE 2
(preferred radicals of the formula A.2):

| | | |
|---|---|---|
| 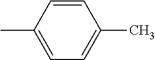 | 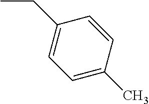 | 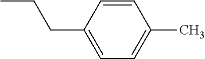 |
| 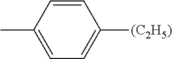 | 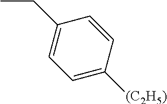 | 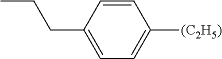 |
| 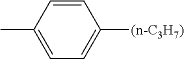 | 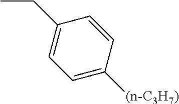 | 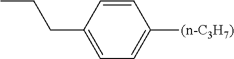 |
| 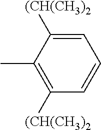 | 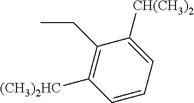 | 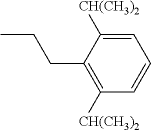 |
| 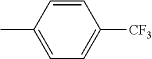 | 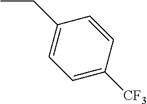 | 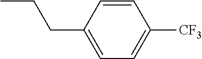 |
| 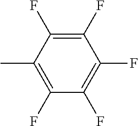 | 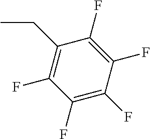 | 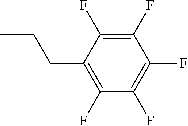 |

In a preferred embodiment R¹, R², R³, R⁴, R⁵, R⁶, R¹¹ and R¹² and, if present, R⁷, R⁸, R⁹ and R¹⁰ are all hydrogen.

In a preferred embodiment, at least one of the radicals R¹, R², R³, R⁴, R⁵, R⁶, R¹¹ and R¹² and, if present, R⁷, R⁸, R⁹ and R¹⁰ is selected from

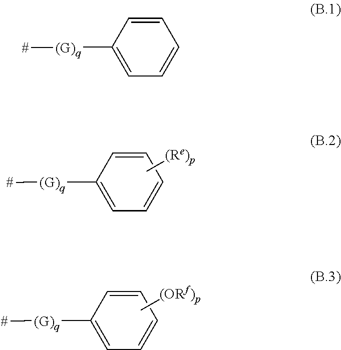

wherein
# represents the bonding side to a nitrogen atom,
G where present, is a divalent bridging group selected from —O—, —S— or C₁-C₁₀-alkylene which may be interrupted and/or terminated by one or more nonadjacent groups which are selected from —O— and —S—,
q is 0 or 1,
R^{e} in formula B.2 is selected independently of one another from C₁-C₃₀-alkyl, C₁-C₃₀-fluoroalkyl, fluorine, chlorine, bromine, NE¹E², nitro and cyano, where E¹ und E², independently of one another, are hydrogen, alkyl, cycloalkyl, heterocycloalkyl, aryl or hetaryl,
R^{f} in formula B.3, is selected independently of one another from C₁-C₃₀-alkyl, p in formulae B.2 and B.3 is 1, 2, 3, 4 or 5.

Examples of preferred radicals of the formula B.1 are mentioned in the following table 3. In a preferred embodiment, in the compounds of the formula (I) at least one of the radicals R¹, R², R³, R⁴, R⁵, R⁶, R¹¹ and R¹² and, if present, R⁷, R⁸, R⁹ and R¹⁰ is selected from radicals of the formula B.1 mentioned in the following table 3.

TABLE 3
(preferred radicals of the formula B.1):

| | | |
|---|---|---|
| 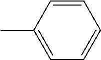 | 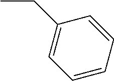 | 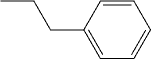 |
| 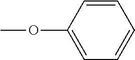 | 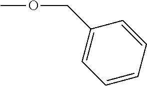 | 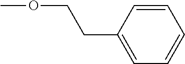 |
| 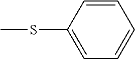 | 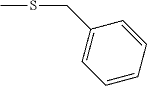 | 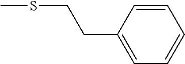 |

Examples of preferred radicals of the formula B.2 are mentioned in the following table 4. In a preferred embodiment, in the compounds of the formula (I) at least one of the radicals R¹, R², R³, R⁴, R⁵, R⁶, R¹¹ and R¹² and, if present, R⁷, R⁸, R⁹ and R¹⁰ is selected from radicals of the formula B.2 mentioned in the following table 4.

TABLE 4
(preferred radicals of the formula B.2):

| | | |
|---|---|---|
| 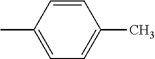 | 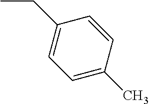 | 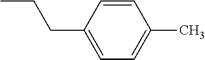 |
| 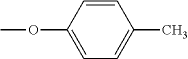 | 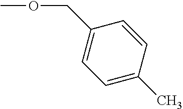 | 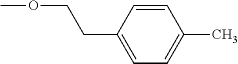 |
| 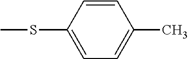 | 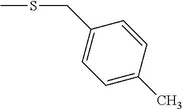 | 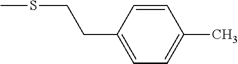 |
| 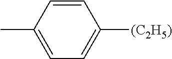 | 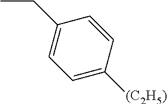 | 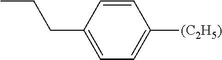 |
| 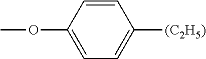 | 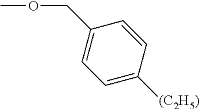 | 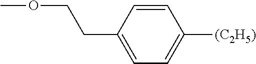 |
| 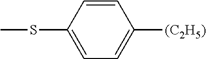 | 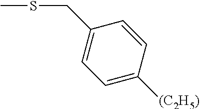 | 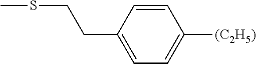 |
| 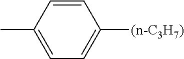 | 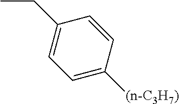 | 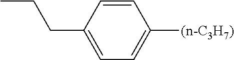 |
| 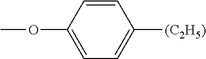 | 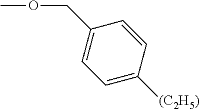 | 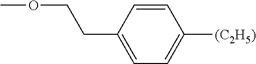 |
| 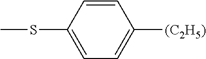 | 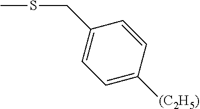 | 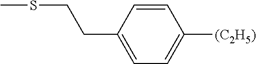 |
| 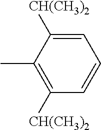 | 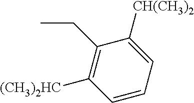 | 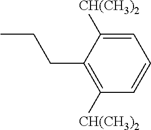 |
| 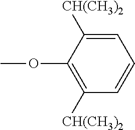 | 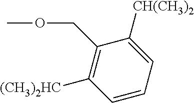 | 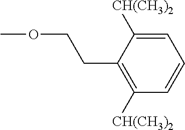 [SMILES: COCCc1c(C(C)C)cccc1C(C)C] |
| 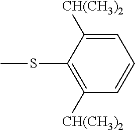 | 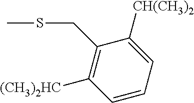 | 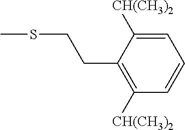 [SMILES: CSCCc1c(C(C)C)cccc1C(C)C] |
| 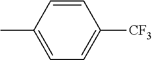 | 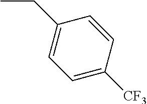 | 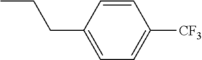 |
| 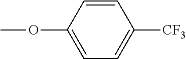 | 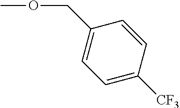 | 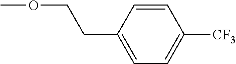 |
| 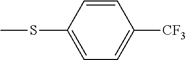 | 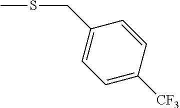 | 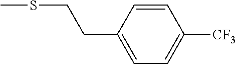 |
| 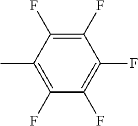 | 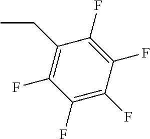 | 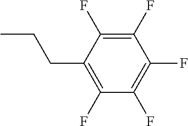 |
| 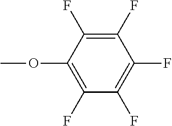 | 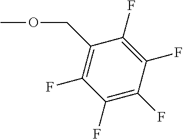 | 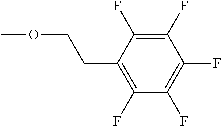 |
| 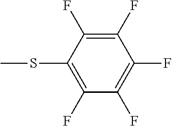 | 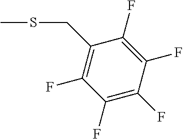 | 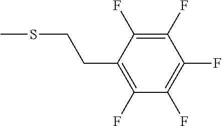 |

In a further preferred embodiment, at least one of the radicals R¹, R², R³, R⁴, R⁵, R⁶, R¹¹ and R¹² and, if present, R⁷, R⁸, R⁹ and R¹⁰ is a linear C₁-C₃₀-alkyl radical. Preferred linear alkyl groups are methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

In a preferred embodiment, at least one of the radicals at least one of the radicals R¹, R², R³, R⁴, R⁵, R⁶, R¹¹ and R¹² and, if present, R⁷, R⁸, R⁹ and R¹⁰ is selected from C₁-C₁₂-alkyl which carries one radical selected from Si(C₁-C₈-alkyl)(O—Si(C₁-C₈-alkyl)₃)₂, Si(phenyl)(O—Si(C₁-C₈-alkyl)₃)₂ and phenyl. More preferably, at least one of the radicals R¹, R², R³, R⁴, R⁵, R⁶, R¹¹ and R¹² and, if present, R⁷, R⁸, R⁹ and R¹⁰ is selected from C₃-C₁₂-alkyl which carries one radical selected from Si(C₁-C₈-alkyl)(O—Si(C₁-C₈-alkyl)₃)₂, Si(phenyl)(O—Si(C₁-C₈-alkyl)₃)₂ and phenyl. In particular, at least one of the radicals R¹, R², R³, R⁴, R⁵, R⁶, R¹¹ and R¹² and, if present, R⁷, R⁸, R⁹ and R¹⁰ is selected from C₃-C₁₂-alkyl, in particular linear C₃-C₁₂-alkyl, which carries one radical selected from Si(C₁-C₄-alkyl)(O—Si(C₁-C₄-alkyl)₃)₂, Si(phenyl)(O—Si(C₁-C₄-alkyl)₃)₂ and phenyl.

In a preferred embodiment, at least one of the radicals at least one of the radicals R¹, R², R³, R⁴, R⁵, R⁶, R¹¹ and R¹² and, if present, R⁷, R⁸, R⁹ and R¹⁰ is selected from [Si(C₁-C₈-alkyl)(O—Si(C₁-C₈-alkyl)₃)₂]-phenylene and [Si(phenyl)(O—Si(C₁-C₈-alkyl)₃)₂]-phenylene.

Some particularly preferred compounds (I) are specified below:

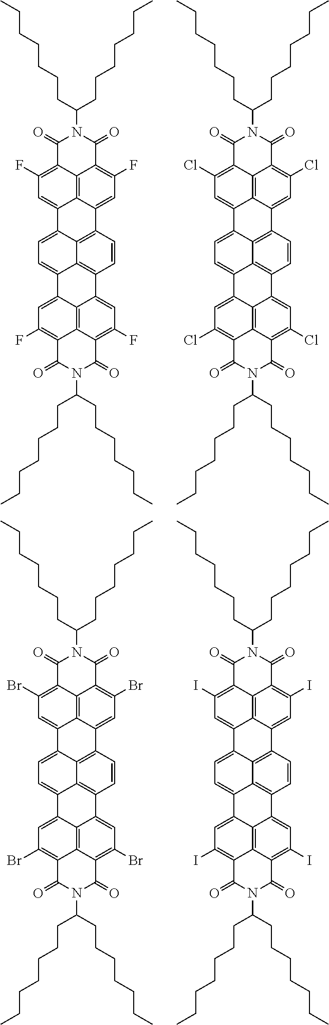

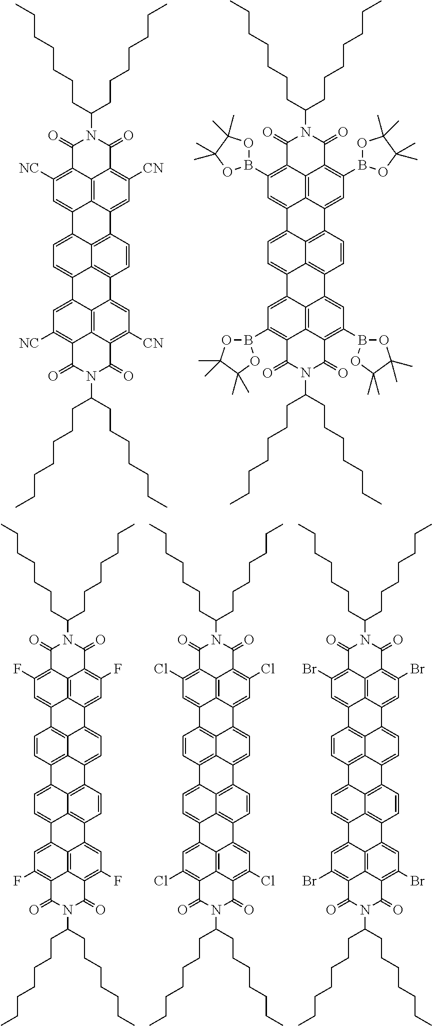

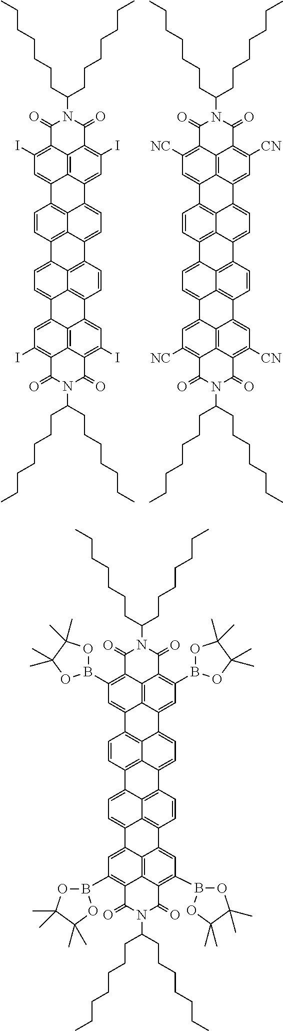

A further object of the invention is a process for the preparation of a compound of the formula I,

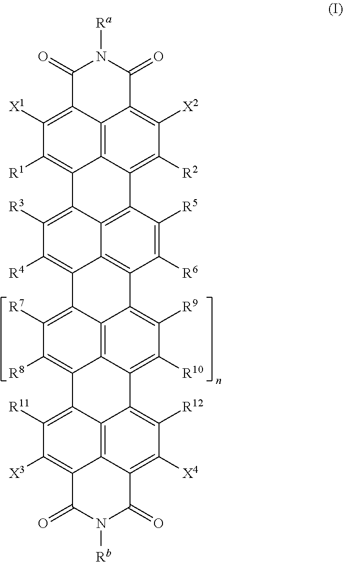

wherein
n is 0 or 1,
R^{a} and R^{b} are independently of one another selected from hydrogen and in each case unsubstituted or substituted alkyl, alkenyl, alkadienyl, alkynyl, cycloalkyl, bicycloalkyl, cycloalkenyl, heterocycloalkyl, aryl or heteroaryl,
X¹, X², X³ and X⁴ are independently of one another selected from F, Cl, Br, I, CN and B(OR^{c})₂,
   wherein R^{c} is selected from in each case unsubstituted or substituted alkyl, cycloalkyl or aryl, or wherein two radicals R^{c} may together form a divalent bridging group selected from in each case unsubstituted or substituted C₂-C₁₀-alkylene, C₃-C₆-cycloalkylene and C₆-C₁₄-arylene, wherein C₂-C₁₀-alkylene, C₃-C₆-cycloalkylene and C₆-C₁₄-arylene may carry one or more identical or different C₁-C₁₂-alkyl radicals,
R¹, R², R³, R⁴, R⁵, R⁶, R¹¹ and R¹² and, if present, R⁷, R⁸, R⁹ and R¹⁰ are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino, (dihetaryl)amino,

comprising
i) reacting a compound of the formula (A) with a diboron compound of the formula (B) in the presence of a transition metal-containing catalyst to obtain a compound of the formula (C)

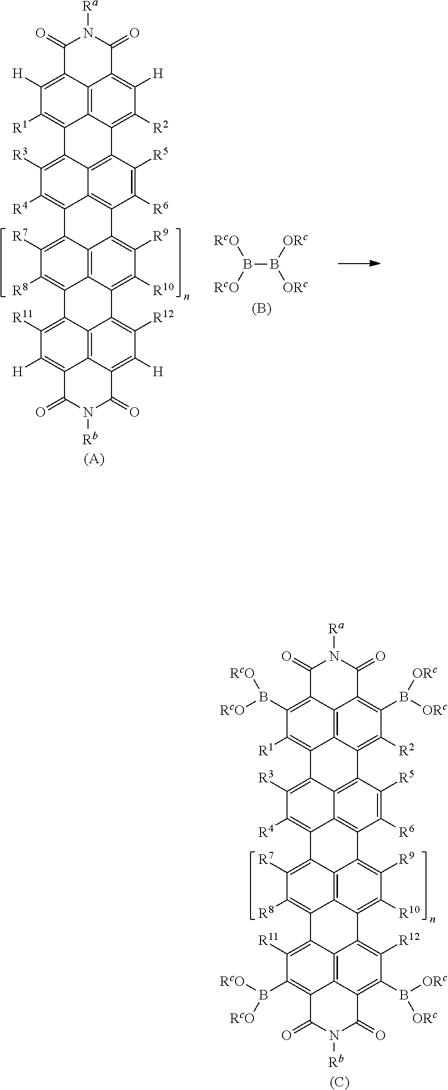

wherein
n, R^{a}, R^{b}, R^{c}, R¹, R², R³, R⁴, R⁵, R⁶, R¹¹ and R¹² and, if present, R⁷, R⁸, R⁹ and R¹⁰, are as defined above, and
ii) optionally reacting the compound of the formula (C) with an F-, Cl-, Br-, I- and/or CN-source to obtain a compound of the formula (I).

Step i)

Suitable compounds of the formula (A) and methods for their preparation are described inter alia in Chem. Mater. 2006, 18 (16), 3715-3725; J. Am. Chem. Soc. 1999, 121 (14), 3513-3520; U.S. Pat. No. 5,986,099; U.S. Pat. No. 6,124,458. The teaching of those documents is incorporated herein by reference.

Preferably, the diboron compound (B) is selected from compounds of the formula (B1)

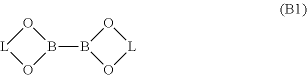

wherein L is selected from unsubstituted linear C₂-C₁₀-alkylene or linear C₂-C₁₀-alkylene substituted by 1, 2, 3, 4, 5 or 6 C₁-C₁₀-alkyl groups.

Preferably, in the compounds (B1) L is selected from unsubstituted linear C₂-C₃-alkylene or linear C₂-C₃-alkylene substituted by 1, 2, 3 or 4 C₁-C₄-alkyl groups.

In particular, the diboron compound (B) is selected from bis(pinacolato)diboron, bis(2,2-dimethyl-1,3-propanediolato)diboron (=bis(neopentyl glycolato)diboron), bis(1,1,3,3-tetramethyl-1,3-propanediolato)diboron, bis(4,5-pinandiolato)diborone and bis(1,2-benzenediolato)diboron. Particular preference is given to bis(pinacolato)diboron.

The transition metal-containing catalyst employed in step i) preferably comprises at least one metal of groups 6-11 of the Periodic Table of the Elements, more preferably at least one metal of groups 8, 9 or 10, in particular at least one metal selected from Ru, Ir, Pd and Pt.

In a first preferred embodiment, the transition metal-containing catalyst employed in step i) comprises a combination of [Ir(OMe)(cod)]₂ and tris(pentafluorophenyl)phosphine as ligand. With regard to suitable reaction conditions for the iridium catalyzed borylation reference is made to Takuro Teraoka, Satoru Hiroto and Hiroshi Shinokubo, Org. Lett. 2011, 13 (10), pp. 2532-2535.

In a second preferred embodiment, the transition metal-containing catalyst employed in step i) is RuH₂(CO)(P(C₆H₅)₃)₃. With regard to suitable reaction conditions for the ruthenium catalyzed borylation, reference is made to Glauco Battagliarin, Yanfei Zhao, Chen Li and Klaus Müllen, Org. Lett. 2011, 13 (13), pp. 3399-3401.

Typically, the transition metal catalyst is used in an amount of from 0.05 to 20% by weight, more preferably from 0.1 to 10% by weight, based on the weight of the compound to be borylated.

The borylation in step i) is preferably carried out at a temperature in the range from 0 to 200° C., more preferably in the range from 10 to 180° C. and in particular in the range from 20 to 150° C.

The borylation in step i) can be carried out in a suitable solvent. Suitable solvents are ones which are inert under the reaction conditions. Suitable solvents include, for example, open-chain and cyclic ethers, e.g. diethyl ether, methyl tert-butyl ether, tetrahydrofuran or 1,4-dioxane; ketones, e.g. acetone, methyl ethyl ketone or pinacolone; alkylbenzenes, e.g. toluene, o-, m- or p-xylene and mesitylene. Mixtures of the abovementioned solvents are also suitable.

In a preferred embodiment, the reaction in step i) is performed under an inert gas atmosphere. Suitable inert gases are for example nitrogen or argon.

Step ii)

In step ii) of the process according to the invention, the boronic ester groups in the 2, 5, and 13 position of the terrylene diimides or the boronic ester groups in the 2, 5, 12 and 15 position of the quaterrylene diimides are replaced with electron withdrawing groups, selected from F, Cl, Br, I and CN.

In a first embodiment, the reaction in step ii) is an exchange of at least a part of the boronic ester groups for fluorine atoms. In particular, in step ii) all the boronic ester groups are replaced with fluorine atoms. The F source employed to introduce the fluorine groups is preferably selected from alkali metal fluorides, in particular KF, NaF or CsF.

Usually the molar ratio of the fluoride source to boronic ester groups of compound (C) is in a range of from 1:1 to 30:1, preferably in a range of from 5:1 to 25:1.

The reaction temperature in step ii) for an exchange of at least a part of the boronic ester groups for fluorine atoms is usually in a range of from 50° C. and 200° C., preferably 100 C to 180° C.

The reaction is usually performed in an aprotic solvent. Suitable aprotic solvents are ethers, such as dioxane and diglyme (bis(2-methoxyethyl)ether), dimethylformamide, N-methylpyrrolidone, (CH₃)₂SO, dimethyl sulfone, sulfolane, cyclic urea such as 1,3-dimethyl-3,4,5,6-tetrahydro-2(1H)-pyrimidinone (DMPU) and imidazolidin-2-one or mixtures thereof.

In a second embodiment, the reaction in step ii) is an exchange of at least a part of the boronic ester groups for chlorine or bromine atoms. In particular, in step ii) all the boronic ester groups are replaced with chlorine atoms, or all the boronic ester groups are replaced with bromine atoms. The halogen source employed to introduce the chlorine or bromine groups is preferably selected from CuCl₂ and CuBr₂.

Preferred solvents for the halogen exchange in step ii) are water, C₁-C₄-alkanols, dioxane, tetrahydrofuran, and mixtures thereof. In particular, a mixture of dioxane, methanol and water is employed as solvent int step ii).

If the reaction in step ii) is an exchange of the boronic ester groups for chlorine atoms or bromine atoms, it can be performed in analogy to the method described by Glauco Battagliarin, Yanfei Zhao, Chen Li and Klaus Müllen in Org. Lett. 2011, 13 (13), pp. 3399-3401.

In a third embodiment, the reaction in step ii) is an exchange of at least a part of the boronic ester groups for iodine atoms. In particular, in step ii) all the boronic ester groups are replaced with iodine atoms. The iodine source employed to introduce the iodine groups is preferably selected from alkali metal iodides, in particular NaI.

In a preferred embodiment, the iodination is performed in the presence of tosylchloramide (chloramine-T).

Preferred solvents for the iodine exchange in step ii) are water, C₁-C₄-alkanols, dioxane, tetrahydrofuran, and mixtures thereof. In particular, a mixture of tetrahydrofuran and water is employed as solvent in step ii).

If the reaction in step ii) is an exchange of the boronic ester groups for iodine atoms, it can be performed in analogy to the method described by Glauco Battagliarin, Chen Li, Volker Enkelmann and Klaus Müllen describe in Org. Lett. 2011, 13 (12), pp. 3012-3015.

In a fourth embodiment, the reaction in step ii) is an exchange of at least a part of the boronic ester groups for cyano groups. In particular, in step ii) all the boronic ester groups are replaced with cyano groups.

Suitable CN sources are selected from the group consisting of tetra-(C₁-C₁₀-alkyl)ammoniumcyanide, tetra-(C₁-C₁₀-alkyl)phosphoniumcyanide, hexa-(C₁-C₁₀)alkylguanidiniumcyanide, Zn(CN)₂ and alkalicyanide.

If the reaction in step ii) is an exchange of the boronic ester groups for cyano groups, it can be performed in analogy to the method described by Glauco Battagliarin, Yanfei Zhao, Chen Li and Klaus Müllen in Org. Lett. 2011, 13 (13), pp. 3399-3401. In principal, if the metal cyanide is Zn(CN)₂, the reaction in step ii) can also be performed in analogy to the method described by C. W. Liskey; X. Liao; J. F. Hartwig in J. Am. Chem. Soc. 2010, 132, 11389-11391.

The compounds of the formula (I) can be isolated by methods known in the art, such as column chromatography.

The compounds of the formula (I) are in particular suitable as organic semiconductors. They generally can function as n-semiconductors or p-semiconductors. In electronic devices that employ a combination of two different semiconductors, e.g. organic solar cells, it depends on the position of the energy levels in the corresponding semiconductor material if a compound of the formula (I) acts as n-semiconductor or as p-semiconductor. Further, if a compound of the formula (I) acts as n-semiconductor or as p-semiconductors depends inter alia on the employed gate dielectric. If the gate dielectric comprises a self-assembled monolayer (SAM) of a fluorine-free compound, e.g. octadecylphosphonic acid, the compounds of the formula (I) usually act as n-semiconductor. If the gate dielectric comprises a self-assembled monolayer (SAM) of a fluorine-containing compound, e.g. 12,12,13,13,14,14,15,15,16,16,17,17,18,18-pentadecafluorooctadecylphosphonic acid, the compounds of the formula (I) usually act as p-semiconductor.

The compounds of the formula (I) have at least one of the following advantages over known organic semiconductor materials:
high charge transport mobility,
air stability,
high on/off ratio,
suitability to be employed in a solvent-based process.

The compounds of the formula (I) are advantageously suitable for organic field-effect transistors. They may be used, for example, for the production of integrated circuits (ICs), for which customary n-channel MOSFETs (metal oxide semiconductor field-effect transistors) have been used to date. These are then CMOS-like semiconductor units, for example for microprocessors, microcontrollers, static RAM and other digital logic circuits. For the production of semiconductor materials, the compounds of the formula (I) can be processed further by one of the following processes: printing (offset, flexographic, gravure, screenprinting, inkjet, electrophotography), evaporation, laser transfer, photolithography, drop-casting. They are especially suitable for use in displays (specifically large-surface area and/or flexible displays), RFID tags, smart labels and sensors.

The compounds of the formula (I) are advantageously suitable as electron conductors in organic field-effect transistors, organic solar cells and in organic light-emitting diodes. They are also particularly advantageous as an exciton transport material in excitonic solar cells.

Some of the compounds of the formula (I) are fluorescent and are also particularly advantageously suitable as fluorescent dyes in a display based on fluorescence conversion. Such displays comprise generally a transparent substrate, a fluorescent dye present on the substrate and a radiation source. Typical radiation sources emit blue (color by blue) or UV light (color by UV). The dyes absorb either the blue or the UV light and are used as green emitters. In these displays, for example, the red light is generated by exciting the red emitter by means of a green emitter which absorbs blue or UV light. Suitable color-by-blue displays are described, for example, in WO 98/28946. Suitable color-by-UV displays are described, for example, by W. A. Crossland, I. D. Sprigle and A. B. Davey in Photoluminescent LCDs (PL-LCD) using phosphors, Cambridge University and Screen Technology Ltd., Cambridge, UK. The compounds of the formula (I) are also particularly suitable in displays which, based on an electrophoretic effect, switch colors on and off via charged pigment dyes. Such electrophoretic displays are described, for example, in US 2004/0130776.

The invention further provides organic field-effect transistors comprising a substrate with at least one gate structure, a source electrode and a drain electrode, and at least one compound of the formula (I) as defined above as a semiconductor.

The invention further provides substrates having a plurality of organic field-effect transistors, wherein at least some of the field-effect transistors comprise at least one compound of the formula (I) as defined above.

The invention also provides semiconductor units which comprise at least one such substrate.

A specific embodiment is a substrate with a pattern (topography) of organic field-effect transistors, each transistor comprising
an organic semiconductor disposed on the substrate;
a gate structure for controlling the conductivity of the conductive channel; and
conductive source and drain electrodes at the two ends of the channel,

the organic semiconductor consisting of at least one compound of the formula (I) or comprising a compound of the formula (I). In addition, the organic field-effect transistor generally comprises a dielectric.

A specific embodiment is a substrate with a pattern (topography) of organic field-effect transistors, each transistor comprising
an organic semiconductor disposed on a buffer layer on a substrate;
a gate structure for controlling the conductivity of the conductive channel; and
conductive source and drain electrodes at the two ends of the channel,

the organic semiconductor consisting of at least one compound of the formula (I) or comprising a compound of the formula (I). In addition, the organic field-effect transistor generally comprises a dielectric.

As a buffer layer, any dielectric material is suitable, for example anorganic materials such LIF, AlOₓ, SiO₂ or silicium nitride or organic materials such as polyimides or polyacrylates, e.g. polymethylmethacrylate (PMMA).

A further specific embodiment is a substrate having a pattern of organic field-effect transistors, each transistor forming an integrated circuit or being part of an integrated circuit and at least some of the transistors comprising at least one compound of the formula (I).

Suitable substrates are in principle the materials known for this purpose. Suitable substrates comprise, for example, metals (preferably metals of groups 8, 9, 10 or 11 of the Periodic Table, such as Au, Ag, Cu), oxidic materials (such as glass, ceramics, SiO₂, especially quartz), semiconductors (e.g. doped Si, doped Ge), metal alloys (for example based on Au, Ag, Cu, etc.), semiconductor alloys, polymers (e.g. polyvinyl chloride, polyolefins, such as polyethylene and polypropylene, polyesters, fluoropolymers, polyamides, polyimides, polyurethanes, polyethersulfones, polyalkyl(meth)acrylates, polystyrene and mixtures and composites thereof), inorganic solids (e.g. ammonium chloride), paper and combinations thereof. The substrates may be flexible or inflexible, and have a curved or planar geometry, depending on the desired use.

A typical substrate for semiconductor units comprises a matrix (for example a quartz or polymer matrix) and, optionally, a dielectric top layer.

Suitable dielectrics are SiO₂, polystyrene, poly-α-methylstyrene, polyolefins (such as polypropylene, polyethylene, polyisobutene), polyvinylcarbazole, fluorinated polymers (e.g. Cytop), cyanopullulans (e.g. CYMM), polyvinylphenol, poly-p-xylene, polyvinyl chloride, or polymers crosslinkable thermally or by atmospheric moisture. Specific dielectrics are “self-assembled nanodielectrics”, i.e. polymers which are obtained from monomers comprising SiCl functionalities, for example Cl₃SiOSiCl₃, Cl₃Si—(CH₂)₆—SiCl₃, Cl₃Si-(CH₂)₁₂—SiCl₃, and/or which are crosslinked by atmospheric moisture or by addition of water diluted with solvents (see, for example, Facchetti, Adv. Mater. 2005, 17, 1705-1725). Instead of water, it is also possible for hydroxyl-containing polymers such as polyvinylphenol or polyvinyl alcohol or copolymers of vinylphenol and styrene to serve as crosslinking components. It is also possible for at least one further polymer to be present during the crosslinking operation, for example polystyrene, which is then also crosslinked (see Facietti, US patent application 2006/0202195).

The substrate may additionally have electrodes, such as gate, drain and source electrodes of OFETs, which are normally localized on the substrate (for example deposited onto or embedded into a nonconductive layer on the dielectric). The substrate may additionally comprise conductive gate electrodes of the OFETs, which are typically arranged below the dielectric top layer (i.e. the gate dielectric).

In a specific embodiment, an insulator layer (gate insulating layer) is present on at least part of the substrate surface. The insulator layer comprises at least one insulator which is preferably selected from inorganic insulators, such as SiO₂, silicon nitride (Si₃N₄), etc., ferroelectric insulators, such as Al₂O₃, Ta₂O₅, La₂O₅, TiO₂, Y₂O₃, etc., organic insulators such as polyimides, benzocyclobutene (BCB), polyvinyl alcohols, polyacrylates, etc., and combinations thereof.

Suitable materials for source and drain electrodes are in principle electrically conductive materials. These include metals, preferably metals of groups 6, 7, 8, 9, 10 or 11 of the Periodic Table, such as Pd, Au, Ag, Cu, Al, Ni, Cr, etc. Also suitable are conductive polymers, such as PEDOT (=poly(3,4-ethylenedioxythiophene)):PSS (=poly(styrenesulfonate)), polyaniline, surface-modified gold, etc. Preferred electrically conductive materials have a specific resistance of less than 10⁻³ ohm×meter, preferably less than 10⁻⁴ ohm×meter, especially less than 10⁻⁶ or 10⁻⁷ ohm×meter.

In a specific embodiment, drain and source electrodes are present at least partly on the organic semiconductor material. It will be appreciated that the substrate may comprise further components as used customarily in semiconductor materials or ICs, such as insulators, resistors, capacitors, conductor tracks, etc.

The electrodes may be applied by customary processes, such as evaporation or sputtering, lithographic processes or another structuring process, such as printing techniques.

The semiconductor materials may also be processed with suitable auxiliaries (polymers, surfactants) in disperse phase by printing.

In a first preferred embodiment, the deposition of at least one compound of the general formula (I) (and if appropriate further semiconductor materials) is carried out by a gas phase deposition process (physical vapor deposition, PVD). PVD processes are performed under high-vacuum conditions and comprise the following steps: evaporation, transport, deposition. It has been found that, surprisingly, the compounds of the general formula (I) are suitable particularly advantageously for use in a PVD process, since they essentially do not decompose and/or form undesired by-products. The material deposited is obtained in high purity. In a specific embodiment, the deposited material is obtained in the form of crystals or comprises a high crystalline content. In general, for the PVD, at least one compound of the general formula (I) is heated to a temperature above its evaporation temperature and deposited on a substrate by cooling below the crystallization temperature. The temperature of the substrate in the deposition is preferably within a range from about 20 to 250° C., more preferably from 50 to 200° C. It has been found that, surprisingly, elevated substrate temperatures in the deposition of the compounds of the formula (I) can have advantageous effects on the properties of the semiconductor elements achieved.

The resulting semiconductor layers generally have a thickness which is sufficient for forming a semiconductor channel which is in contact with the source/drain electrodes. The deposition can be effected under an inert atmosphere, for example, under nitrogen, argon or helium.

The deposition is effected typically at ambient pressure or under reduced pressure. A suitable pressure range is from about 10⁻⁷ to 1.5 bar.

The compound of the formula (I) is preferably deposited on the substrate in a thickness of from 10 to 1000 nm, more preferably from 15 to 250 nm. In a specific embodiment, the compound of the formula (I) is deposited at least partly in crystalline form. For this purpose, especially the above-described PVD process is suitable. Moreover, it is possible to use previously prepared organic semiconductor crystals. Suitable processes for obtaining such crystals are described by R. A. Laudise et al. in “Physical Vapor Growth of Organic Semi-Conductors”, Journal of Crystal Growth 187 (1998), pages 449-454, and in “Physical Vapor Growth of Centimeter-sized Crystals of α-Hexathiophene”, Journal of Crystal Growth 1982 (1997), pages 416-427, which are incorporated here by reference.

In a second preferred embodiment, the deposition of at least one compound of the general formula (I) (and if appropriate further semiconductor materials) is effected by spin-coating. Surprisingly, it is thus also possible to use the compounds of the formula (I) used in accordance with the invention in a wet processing method to produce semiconductor substrates. The compounds of the formula (I) should thus also be suitable for producing semiconductor elements, especially OFETs or based on OFETs, by a printing process. It is possible for this purpose to use customary printing or coating processes (inkjet, flexographic, offset, gravure; intaglio printing, nanoprinting, slot die). Preferred solvents for the use of compounds of the formula (I) in a printing process are aromatic solvents, such as toluene, xylene, etc. It is also possible to add thickening substances, such as polymers, for example polystyrene, etc., to these “semiconductor inks”. In this case, the dielectrics used are the aforementioned compounds.

In a preferred embodiment, the inventive field-effect transistor is a thin-film transistor (TFT). In a customary construction, a thin-film transistor has a gate electrode disposed on the substrate or buffer layer (the buffer layer being part of the substrate), a gate insulation layer disposed thereon and on the substrate, a semiconductor layer disposed on the gate insulator layer, an ohmic contact layer on the semiconductor layer, and a source electrode and a drain electrode on the ohmic contact layer.

In a preferred embodiment, the surface of the substrate, before the deposition of at least one compound of the general formula (I) (and if appropriate of at least one further semiconductor material), is subjected to a modification. This modification serves to form regions which bind the semiconductor materials and/or regions on which no semiconductor materials can be deposited. The surface of the substrate is preferably modified with at least one compound (C1) which is suitable for binding to the surface of the substrate and to the compounds of the formula (I). In a suitable embodiment, a portion of the surface or the complete surface of the substrate is coated with at least one compound (C1) in order to enable improved deposition of at least one compound of the general formula (I) (and if appropriate further semiconductive compounds). A further embodiment comprises the deposition of a pattern of compounds of the general formula (C1) on the substrate by a corresponding production process. These include the mask processes known for this purpose and so-called “patterning” processes, as described, for example, in U.S. Ser. No. 11/353,934, which is incorporated here fully by reference.

Suitable compounds of the formula (C1) are capable of a binding interaction both with the substrate and with at least one semiconductor compound of the general formula (I). The term “binding interaction” comprises the formation of a chemical bond (covalent bond), ionic bond, coordinative interaction, van der Waals interactions, e.g. dipole-dipole interactions etc.), and combinations thereof. Suitable compounds of the general formula (C1) are:
silane, phosphonic acids, carboxylic acids, hydroxamic acids, such as alkyltrichlorosilanes, e.g. n-octadecyltrichlorosilane; compounds with trialkoxysilane groups, e.g. alkyltrialkoxysilanes such as n-octadecyltrimethoxysilane, n-octadecyltriethoxysilane, n-octadecyltri(n-propyl)oxysilane, noctadecyltri(isopropyl)oxysilane; trialkoxyaminoalkylsilanes, such as triethoxyaminopropylsilane and N[(3-triethoxysilyl)propyl]ethylenediamine; trialkoxyalkyl 3-glycidyl ether silanes, such as triethoxypropyl 3-glycidyl ether silane; trialkoxyallylsilanes, such as allyltrimethoxysilane; trialkoxy(isocyanatoalkyl)silanes; trialkoxysilyl(meth)acryloyloxyalkanes and trialkoxysilyl(meth)acrylamidoalkanes, such as 1-triethoxysilyl-3-acryl-oyl-oxypropane.
amines, phosphines and sulfur-comprising compounds, especially thiols.

The compound (C1) is preferably selected from alkyltrialkoxysilanes, especially n-octadecyltrimethoxysilane, n-octadecyltriethoxysilane; hexaalkyldisilazanes, and especially hexamethyldisilazane (HMDS); C₈-C₃₀-alkylthiols, especially hexadecanethiol; mercaptocarboxylic acids and mercaptosulfonic acids, especially mercaptoacetic acid, 3-mercaptopropionic acid, mercaptosuccinic acid, 3-mercapto-1-propanesulfonic acid and the alkali metal and ammonium salts thereof.

Various semiconductor architectures comprising the inventive semiconductors are also conceivable, for example top contact, top gate, bottom contact, bottom gate, or else a vertical construction, for example a VOFET (vertical organic field-effect transistor), as described, for example, in US 2004/0046182.

Preferred semiconductor architectures are the following, depicted in FIG. 3:
1. substrate, dielectric, organic semiconductor, preferably gate, dielectric, organic semiconductor, source and drain, known as “Bottom Gate Top Contact”;
2. substrate, dielectric, organic semiconductor, preferably substrate, gate, dielectric, source and drain, organic semiconductor, known as “Bottom Gate Bottom Contact”;
3. substrate, organic semiconductor, dielectric, preferably substrate, source and drain, organic semiconductor, dielectric, gate, known as “Top Gate Bottom Contact”;
4. substrate, organic semiconductor, dielectric, preferably substrate, organic semiconductor, source and drain, dielectric, gate, known as “Top Gate Top Contact”;

The layer thicknesses are, for example, from 10 nm to 5 μm in semiconductors, from 50 nm to 10 μm in the dielectric; the electrodes may, for example, be from 20 nm to 10 μm. The OFETs may also be combined to form other components, such as ring oscillators or inverters.

A further aspect of the invention is the provision of electronic components which comprise a plurality of semiconductor components, which may be n- and/or p-semiconductors. Examples of such components are field-effect transistors (FETs), bipolar junction transistors (BJTs), tunnel diodes, converters, light-emitting components, biological and chemical detectors or sensors, temperature-dependent detectors, photodetectors, such as polarization-sensitive photodetectors, gates, AND, NAND, NOT, OR, TOR and NOR gates, registers, switches, timer units, static or dynamic stores and other dynamic or sequential, logical or other digital components including programmable switches.

A specific semiconductor element is an inverter. In digital logic, the inverter is a gate which inverts an input signal. The inverter is also referred to as a NOT gate. Real inverter switches have an output current which constitutes the opposite of the input current. Typical values are, for example, (0, +5V) for TTL switches. The performance of a digital inverter reproduces the voltage transfer curve (VTC), i.e. the plot of input current against output current. Ideally, it is a staged function and, the closer the real measured curve approximates to such a stage, the better the inverter is. In a specific embodiment of the invention, the compounds of the formula (I) are used as organic semiconductors in an inverter.

The compounds of the formula (I) are also particularly advantageously suitable for use in organic photovoltaics (OPVs). Preference is given to their use in solar cells which are characterized by diffusion of excited states (exciton diffusion). In this case, one or both of the semiconductor materials utilized is notable for a diffusion of excited states (exciton mobility). Also suitable is the combination of at least one semiconductor material which is characterized by diffusion of excited states with polymers which permit conduction of the excited states along the polymer chain. In the context of the invention, such solar cells are referred to as excitonic solar cells. The direct conversion of solar energy to electrical energy in solar cells is based on the internal photo effect of a semiconductor material, i.e. the generation of electron-hole pairs by absorption of photons and the separation of the negative and positive charge carriers at a p-n transition or a Schottky contact. An exciton can form, for example, when a photon penetrates into a semiconductor and excites an electron to transfer from the valence band into the conduction band. In order to generate current, the excited state generated by the absorbed photons must, however, reach a p-n transition in order to generate a hole and an electron which then flow to the anode and cathode. The photovoltage thus generated can bring about a photocurrent in an external circuit, through which the solar cell delivers its power. The semiconductor can absorb only those photons which have an energy which is greater than its band gap. The size of the semiconductor band gap thus determines the proportion of sunlight which can be converted to electrical energy. Solar cells consist normally of two absorbing materials with different band gaps in order to very effectively utilize the solar energy. Most organic semiconductors have exciton diffusion lengths of up to 10 nm. There is still a need here for organic semiconductors through which the excited state can be passed on over very large distances. It has now been found that, surprisingly, the compounds of the general formula (I) described above are particularly advantageously suitable for use in excitonic solar cells.

Organic solar cells generally have a layer structure and generally comprise at least the following layers: anode, photoactive layer and cathode. These layers are generally applied to a substrate suitable for this purpose. The structure of organic solar cells is described, for example, in US 2005/0098726 and US 2005/0224905.

The invention provides an organic solar cell which comprises a substrate with at least one cathode and at least one anode, and at least one compound of the general formula (I) as defined above as a photoactive material. The inventive organic solar cell comprises at least one photoactive region. A photoactive region may comprise two layers, each of which has a homogeneous composition and forms a flat donor-acceptor heterojunction. A photoactive region may also comprise a mixed layer and form a donor-acceptor heterojunction in the form of a donor-acceptor bulk heterojunction. Organic solar cells with photoactive donor-acceptor transitions in the form of a bulk heterojunction are a preferred embodiment of the invention.

Suitable substrates for organic solar cells are, for example, oxidic materials, polymers and combinations thereof. Preferred oxidic materials are selected from glass, ceramic, SiO₂, quartz, etc. Preferred polymers are selected from polyethylene terephthalates, polyolefins (such as polyethylene and polypropylene), polyesters, fluoropolymers, polyamides, polyurethanes, polyalkyl(meth)acrylates, polystyrenes, polyvinyl chlorides and mixtures and composites.

Suitable electrodes (cathode, anode) are in principle metals, semiconductors, metal alloys, semiconductor alloys, nanowire thereof and combinations thereof. Preferred metals are those of groups 2, 8, 9, 10, 11 or 13 of the periodic table, e.g. Pt, Au, Ag, Cu, Al, In, Mg or Ca. Preferred semiconductors are, for example, doped Si, doped Ge, indium tin oxide (ITO), fluorinated tin oxide (FTO), gallium indium tin oxide (GITO), zinc indium tin oxide (ZITO), poly(3,4-ethylenedioxythiophene)poly(styrenesulfonate) (PEDOT-PSS), etc. Preferred metal alloys are, for example, alloys based on Pt, Au, Ag, Cu, etc. A specific embodiment is Mg/Ag alloys.

The material used for the electrode facing the light (the anode in a normal structure, the cathode in an inverse structure) is preferably a material at least partly transparent to the incident light. This preferably includes electrodes which have glass and/or a transparent polymer as a carrier material. Transparent polymers suitable as carriers are those mentioned above, such as polyethylene terephthalate. The electrical contact connection is generally effected by means of metal layers and/or transparent conductive oxides (TCOs). These preferably include ITO, doped ITO, FTO (fluorine doped tin oxide), AZO (aluminum doped tin oxide), ZnO, TiO₂, Ag, Au, Pt. Particular preference is given to ITO for contact connection. For electrical contact connection, it is also possible to use a conductive polymer, for example a poly-3,4-alkylenedioxythiophene, e.g. poly-3,4-ethyleneoxythiophene poly(styrenesulfonate) (PEDOT).

The electrode facing the light is configured such that it is sufficiently thin to bring about only minimal light absorption but thick enough to enable good charge transport of the extracted charge carriers. The thickness of the electrode layer (without carrier material) is preferably within a range from 20 to 200 nm.

In a specific embodiment, the material used for the electrode facing away from the light (the cathode in a normal structure, the anode in an inverse structure) is a material which at least partly reflects the incident light. This includes metal films, preferably of Ag, Au, Al, Ca, Mg, In, and mixtures thereof. Preferred mixtures are Mg/Al. The thickness of the electrode layer is preferably within a range from 20 to 300 nm.

The photoactive region comprises or consists of at least one layer which comprises at least one compound of the general formula (I) as defined above. In addition, the photoactive region may have one or more further layer(s). These are, for example, selected from
layers with electron-conducting properties (electron transport layer, ETL),
layers which comprise a hole-conducting material (hole transport layer, HTL), which need not absorb any radiation,
exciton- and hole-blocking layers (e.g. EBLs), which must not absorb, and multiplication layers.

Suitable materials for these layers are described in detail hereinafter.

Suitable exciton- and hole-blocking layers are described, for example, in U.S. Pat. No. 6,451,415.

Suitable materials for exciton-blocking layers are, for example, bathocuproin (BCP), 4,4′,4″-tris[3-methylphenyl-N-phenylamino]triphenylamine (m-MTDATA).

The inventive solar cells comprise at least one photoactive donor-acceptor heterojunction. Optical excitation of an organic material generates excitons. In order that a photocurrent occurs, the electron-hole pair has to be separated, typically at a donor-acceptor interface between two unlike contact materials. At such an interface, the donor material forms a heterojunction with an acceptor material. When the charges are not separated, they can recombine in a process also known as “quenching”, either radiatively by the emission of light of a lower energy than the incident light or nonradiatively by generation of heat. Both processes are undesired. According to the invention, at least one compound of the general formula (I) can be used as a charge generator (donor) or as electron acceptor material.

If at least one compound of the general formula (I) is used as a charge generator (donor) it can be combined with an appropriate electron acceptor material (ETM, electron transport material). Radiative excitation is followed by a rapid electron transfer to the ETM. Suitable ETMs are, for example, C60 and other fullerenes, perylene-3,4;9,10-bis(dicarboximides) (PTCDIs), or n-doped layers thereof (as described hereinafter). Preferred ETMs are C60 and other fullerenes or n-doped layers thereof.

In a first embodiment, the heterojunction has a flat configuration (see: Two layer organic photovoltaic cell, C. W. Tang, Appl. Phys. Lett., 48 (2), 183-185 (1986) or N. Karl, A. Bauer, J. Holzapfel, J. Marktanner, M. Möbus, F. Stilzle, Mol. Cryst. Liq. Cryst., 252, 243-258 (1994).).

In a second preferred embodiment, the heterojunction is configured as a bulk (mixed) heterojunction, also referred to as an interpenetrating donor-acceptor network. Organic photovoltaic cells with a bulk heterojunction are described, for example, by C. J. Brabec, N. S. Sariciftci, J. C. Hummelen in Adv. Funct. Mater., 11 (1), 15 (2001) or by J. Xue, B. P. Rand, S. Uchida and S. R. Forrest in J. Appl. Phys. 98, 124903 (2005). Bulk heterojunctions are discussed in detail hereinafter.

The compounds of the formula (I) can be used as a photoactive material in cells with MiM, pin, pn, Mip or Min structure (M=metal, p=p-doped organic or inorganic semiconductor, n=n-doped organic or inorganic semiconductor, i=intrinsically conductive system of organic layers; see, for example, J. Drechsel et al., Org. Electron., 5 (4), 175 (2004) or Maennig et al., Appl. Phys. A 79, 1-14 (2004)).

The compounds of the formula (I) can also be used as a photoactive material in tandem cells. Suitable tandem cells are described, for example, by P. Peumans, A. Yakimov, S. R. Forrest in J. Appl. Phys., 93 (7), 3693-3723 (2003) (see also U.S. Pat. No. 4,461,922, U.S. Pat. No. 6,198,091 and U.S. Pat. No. 6,198,092) and are described in detail hereinafter. The use of compounds of the general formula (I) in tandem cells is a preferred embodiment of the invention.

The compounds of the formula (I) can also be used as a photoactive material in tandem cells which are constructed from two or more than two stacked MiM, pin, Mip or Min structures (see DE 103 13 232.5 and J. Drechsel et al., Thin Solid Films, 451452, 515-517 (2004)).

The layer thickness of the M, n, i and p layers is typically within a range from 10 to 1000 nm, more preferably from 10 to 400 nm. The layers which form the solar cell can be produced by customary processes known to those skilled in the art. These include vapor deposition under reduced pressure or in an inert gas atmosphere, laser ablation or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

In order to improve the efficiency of organic solar cells, it is possible to shorten the mean distance through which the exciton has to diffuse in order to arrive at the next donor-acceptor interface. To this end, it is possible to use mixed layers of donor material and acceptor material which form an interpenetrating network in which internal donor-acceptor heterojunctions are possible. This bulk heterojunction is a specific form of the mixed layer, in which the excitons generated need only travel a very short distance before they arrive at a domain boundary, where they are separated.

In a preferred embodiment, the photoactive donor-acceptor transitions in the form of a bulk heterojunction are produced by a gas phase deposition process (physical vapor deposition, PVD). Suitable processes are described, for example, in US 2005/0227406, to which reference is made here. To this end, a compound of the general formula (I) and a complementary semiconductor material can be subjected to a gas phase deposition in the manner of a cosublimation. PVD processes are performed under high-vacuum conditions and comprise the following steps: evaporation, transport, deposition. The deposition is effected preferably at a pressure within a range from about 10⁻² mbar to 10⁻⁷ mbar, for example from 10⁻⁵ to 10⁻⁷ mbar. The deposition rate is preferably within a range from 0.01 to 100 nm/s. The deposition can be effected in an inert gas atmosphere, for example under nitrogen, helium or argon. The temperature of the substrate during the deposition is preferably within a range from −100 to 300° C., more preferably from −50 to 250° C.

The other layers of the organic solar cell can be produced by known processes. These include vapor deposition under reduced pressure or in an inert gas atmosphere, laser ablation, or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

The photoactive layer (homogeneous layer or mixed layer) can be subjected to a thermal treatment directly after production thereof or after production of further layers which form the solar cell. Such a heat treatment can in many cases further improve the morphology of the photoactive layer. The temperature is preferably within a range from about 60° C. to 300° C. The treatment time is preferably within a range from 1 minute to 3 hours. In addition or alternatively to a thermal treatment, the photoactive layer (mixed layer) can be subjected to a treatment with a solvent-containing gas directly after production thereof or after production of further layers which form the solar cell. In a suitable embodiment, saturated solvent vapors in air are used at ambient temperature.

Suitable solvents are toluene, xylene, chloroform, N-methylpyrrolidone, dimethylformamide, ethyl acetate, chlorobenzene, dichloromethane and mixtures thereof. The treatment time is preferably within a range from 1 minute to 3 hours.

In a suitable embodiment, the inventive solar cells are present as an individual cell with flat heterojunction and normal structure. In a specific embodiment, the cell has the following structure:
an at least partly transparent conductive layer (top electrode, anode) (11)
a hole-conducting layer (hole transport layer, HTL) (12)
a layer which comprises a donor material (13)
a layer which comprises an acceptor material (14)
an exciton-blocking and/or electron-conducting layer (15)
a second conductive layer (back electrode, cathode) (16)

The donor material preferably comprises at least one compound of the formula (I) or consists of a compound of the formula (I). The acceptor material preferably comprises at least one fullerene or fullerene derivative, or consists of a fullerene or fullerene derivative. The acceptor material preferably comprises C60 or PCBM ([6,6]-phenyl-C61-butyric acid methyl ester).

The essentially transparent conductive layer (11) (anode) comprises a carrier, such as glass or a polymer (e.g. polyethylene terephthalate) and a conductive material, as described above. Examples include ITO, doped ITO, FTO, ZnO, AZO, etc. The anode material can be subjected to a surface treatment, for example with UV light, ozone, oxygen plasma, Br₂, etc. The layer (11) should be sufficiently thin to enable maximum light absorption, but also sufficiently thick to ensure good charge transport. The layer thickness of the transparent conductive layer (11) is preferably within a range from 20 to 200 nm.

Solar cells with normal structure optionally have a hole-conducting layer (HTL). This layer comprises at least one hole-conducting material (hole transport material, HTM). Layer (12) may be an individual layer of essentially homogeneous composition or may comprise two or more than two sublayers.

Hole-conducting materials (HTM) suitable for forming layers with hole-conducting properties (HTL) preferably comprise at least one material with high ionization energy.

The ionization energy is preferably at least 5.0 eV, more preferably at least 5.5 eV. The materials may be organic or inorganic materials. Organic materials suitable for use in a layer with hole-conducting properties are preferably selected from poly(3,4-ethylenedioxythiophene)poly(styrenesulfonate) (PEDOT-PSS), Ir-DPBIC (tris-N,N′-diphenylbenzimidazol-2-ylideneiridium(III)), N,N′-diphenyl-N,N′-bis(3-methylphenyl)-1,1′-diphenyl-4,4′-diamine (α-NPD), 2,2′,7,7′-tetrakis(N,N-di-p-methoxyphenylamine)-9,9′-spirobifluorene (spiro-MeOTAD), etc. and mixtures thereof. The organic materials may, if desired, be doped with a p-dopant which has a LUMO within the same range as or lower than the HOMO of the hole-conducting material. Suitable dopants are, for example, 2,3,5,6-tetrafluoro-7,7,8,8-tetracyanoquinodimethane (F₄TCNQ), WO₃, MoO₃, etc. Inorganic materials suitable for use in a layer with hole-conducting properties are preferably selected from WO₃, MoO₃, etc.

If present, the thickness of the layers with hole-conducting properties is preferably within a range from 5 to 200 nm, more preferably 10 to 100 nm.

Layer (13) comprises at least one compound of the general formula (I). The thickness of the layer should be sufficient to absorb a maximum amount of light, but thin enough to enable effective dissipation of the charge. The thickness of the layer (13) is preferably within a range from 5 nm to 1 μm, more preferably from 5 to 100 nm.

Layer (14) comprises at least one acceptor material. The acceptor material preferably comprises at least one fullerene or fullerene derivative. Alternatively or additionally suitable acceptor materials are specified hereinafter. The thickness of the layer should be sufficient to absorb a maximum amount of light, but thin enough to enable effective dissipation of the charge. The thickness of the layer (14) is preferably within a range from 5 nm to 1 μm, more preferably from 5 to 80 nm.

Solar cells with normal structure optionally comprise an exciton-blocking and/or electron-conducting layer (15) (EBL/ETL). Suitable materials for exciton-blocking layers generally have a greater band gap than the materials of layer (13) and/or (14). They are firstly capable of reflecting excitons and secondly enable good electron transport through the layer. The materials for the layer (15) may comprise organic or inorganic materials. Suitable organic materials are preferably selected from 2,9-dimethyl-4,7-diphenyl-1,10-phenanthroline (BCP), 4,7-diphenyl-1,10-phenanthroline (Bphen), 1,3-bis[2-(2,2′bipyridin-6-yl)-1,3,4-oxadiazo-5-yl]benzene (BPY-OXD), etc. The organic materials may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. Inorganic materials suitable for use in a layer with electron-conducting properties are preferably selected from ZnO, etc. If present, the thickness of the layer (15) is preferably within a range from 5 to 500 nm, more preferably 10 to 100 nm.

Layer 16 is the cathode and preferably comprises at least one compound with low work function, more preferably a metal such as Ag, Al, Mg, Ca, etc. The thickness of the layer (16) is preferably within a range from about 10 nm to 10 μm, e.g. 10 nm to 60 nm.

In a further suitable embodiment, the inventive solar cells are present as an individual cell with a flat heterojunction and inverse structure.

In a specific embodiment, the cell has the following structure:
an at least partly transparent conductive layer (cathode) (11)
an exciton-blocking and/or electron-conducting layer (12)
a layer which comprises an acceptor material (13)
a layer which comprises a donor material (14)
a hole-conducting layer (hole transport layer, HTL) (15)
a second conductive layer (back electrode, anode) (16)

With regard to suitable and preferred materials for the layers (11) to (16), reference is made to the above remarks regarding the corresponding layers in solar cells with normal structure.

In a further preferred embodiment, the inventive solar cells are present as an individual cell with normal structure and have a bulk heterojunction. In a specific embodiment, the cell has the following structure:
an at least partly transparent conductive layer (anode) (21)
a hole-conducting layer (hole transport layer, HTL) (22)
a mixed layer which comprises a donor material and an acceptor material, which form a donor-acceptor heterojunction in the form of a bulk heterojunction (23)
an electron-conducting layer (24)
an exciton-blocking and/or electron-conducting layer (25)
a second conductive layer (back electrode, cathode) (26)

The layer (23) comprises at least one compound of the general formula (I) as a photoactive material, e.g. as a donor material. The layer (23) additionally comprises a complementary semiconductor material, e.g. at least one fullerene or fullerene derivative as an acceptor material. The layer (23) comprises especially C60 or PCBM ([6,6]-phenyl-C61-butyric acid methyl ester) as an acceptor material.

With regard to layer (21), reference is made completely to the above remarks regarding layer (11).

With regard to layer (22), reference is made completely to the above remarks regarding layer (12).

Layer (23) is a mixed layer which comprises at least one compound of the general formula (I) as a semiconductor material. In addition, layer (23) comprises at least one complementary semiconductor material. As described above, the layer (23) can be produced by coevaporation or by solution processing using customary solvents. The mixed layer comprises preferably 10 to 90% by weight, more preferably 20 to 80% by weight, of at least one compound of the general formula (I), based on the total weight of the mixed layer. The mixed layer comprises preferably 10 to 90% by weight, more preferably 20 to 80% by weight, of at least one acceptor material, based on the total weight of the mixed layer. The thickness of the layer (23) should be sufficient to absorb a maximum amount of light, but thin enough to enable effective dissipation of the charge. The thickness of the layer (23) is preferably within a range from 5 nm to 1 μm, more preferably from 5 to 200 nm, especially 5 to 80 nm.

Solar cells with a bulk heterojunction comprise an electron-conducting layer (24) (ETL). This layer comprises at least one electron transport material (ETM). Layer (24) may be a single layer of essentially homogeneous composition or may comprise two or more than two sublayers. Suitable materials for electron-conducting layers generally have a low work function or ionization energy. The ionization energy is preferably not more than 3.5 eV. Suitable organic materials are preferably selected from the aforementioned fullerenes and fullerene derivatives, 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials used in layer (24) may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. The thickness of the layer (23) is, if present, preferably within a range from 1 nm to 1 μm, particularly 5 to 60 nm.

With regard to layer (25), reference is made completely to the above remarks regarding layer (15).

With regard to layer (26), reference is made completely to the above remarks regarding layer (16).

Solar cells with a donor-acceptor heterojunction in the form of a bulk heterojunction can be produced by a gas phase deposition process as described above. With regard to deposition rates, substrate temperature during the deposition and thermal aftertreatment, reference is made to the above remarks.

In a further preferred embodiment, the inventive solar cells are present as an individual cell with inverse structure and have a bulk heterojunction.

In a particularly preferred embodiment, the inventive solar cell is a tandem cell.

A tandem cell consists of two or more than two (e.g. 3, 4, 5, etc.) subcells. A single subcell, some of the subcells or all subcells may have photoactive donor-acceptor heterojunctions. Each donor-acceptor heterojunction may be in the form of a flat heterojunction or in the form of a bulk heterojunction. Preferably, at least one of the donor-acceptor heterojunctions is in the form of a bulk heterojunction. According to the invention, the photoactive layer of at least one subcell comprises a compound of the general formula (I). Preferably, the photoactive layer of at least one subcell comprises a compound of the general formula (I) and at least one fullerene or fullerene derivative. More preferably, the semiconductor mixture used in the photoactive layer of at least one subcell consists of a compound of the general formula (I) and C₆₀ or [6,6]-phenyl-C61-butyric acid methyl ester.

The subcells which form the tandem cell may be connected in parallel or in series. The subcells which form the tandem cell are preferably connected in series. There is preferably an additional recombination layer in each case between the individual subcells. The individual subcells have the same polarity, i.e. generally either only cells with normal structure or only cells with inverse structure are combined with one another.

The inventive tandem cell preferably comprises a transparent conductive layer (layer 31). Suitable materials are those specified above for the individual cells. Layers 32 and 34 constitute subcells. “Subcell” refers here to a cell as defined above without cathode and anode. The subcells may, for example, either all have a compound of the general formula (I) used in accordance with the invention in the photoactive layer (preferably in combination with a fullerene or fullerene derivative, especially C60) or have other combinations of semiconductor materials, for example C60 with zinc phthalocyanine, C60 with oligothiophene (such as DCV5T). In addition, individual subcells may also be configured as dye-sensitized solar cells or polymer cells.

In all cases, preference is given to a combination of materials which exploit different regions of the spectrum of the incident light, for example of natural sunlight. For instance, the combination of a compound of the general formula (I) and fullerene or fullerene derivative used in accordance with the invention absorbs in the long-wave region of sunlight. Cells based on at least one perylene compound as described, for example, in International patent application WO2011158211, absorb primarily in the short-wave range. Thus, a tandem cell composed of a combination of these subcells should absorb radiation in the range from about 400 nm to 900 nm. Suitable combination of subcells should thus allow the spectral range utilized to be extended. For optimal performance properties, optical interference should be considered. For instance, subcells which absorb at relatively short wavelengths should be arranged closer to the metal top contact than subcells with longer-wave absorption.

With regard to layer (31), reference is made completely to the above remarks regarding layers (11) and (21).

With regard to layers (32) and (34), reference is made completely to the above remarks regarding layers (12) to (15) for flat heterojunctions and (22) to (25) for bulk heterojunctions.

Layer 33 is a recombination layer. Recombination layers enable the charge carriers from one subcell to recombine with those of an adjacent subcell. Small metal clusters are suitable, such as Ag, Au or combinations of highly n- and p-doped layers. In the case of metal clusters, the layer thickness is preferably within a range from 0.5 to 5 nm. In the case of highly n- and p-doped layers, the layer thickness is preferably within a range from 5 to 40 nm. The recombination layer generally connects the electron-conducting layer of a subcell to the hole-conducting layer of an adjacent subcell. In this way, further cells can be combined to form the tandem cell.

Layer 36 is the top electrode. The material depends on the polarity of the subcells. For subcells with normal structure, preference is given to using metals with a low work function, such as Ag, Al, Mg, Ca, etc. For subcells with inverse structure, preference is given to using metals with a high work function, such as Au or Pt, or PEDOT-PSS.

In the case of subcells connected in series, the overall voltage corresponds to the sum of the individual voltages of all subcells. The overall current, in contrast, is limited by the lowest current of one subcell. For this reason, the thickness of each subcell should be optimized such that all subcells have essentially the same current.

Examples of different kinds of donor-acceptor heterojunctions are a donor-acceptor double layer with a flat heterojunction, or the heterojunction is configured as a hybrid planar-mixed heterojunction or gradient bulk heterojunction or annealed bulk heterojunction.

The production of a hybrid planar-mixed heterojunction is described in Adv. Mater. 17, 66-70 (2005). In this structure, mixed heterojunction layers which were formed by simultaneous evaporation of acceptor and donor material are present between homogeneous donor and acceptor material.

In a specific embodiment of the present invention, the donor-acceptor-heterojunction is in the form of a gradient bulk heterojunction. In the mixed layers composed of donor and acceptor materials, the donor-acceptor ratio changes gradually. The form of the gradient may be stepwise or linear. In the case of a stepwise gradient, the layer 01 consists, for example, of 100% donor material, layer 02 has a donor/acceptor ratio>1, layer 03 has a donor/acceptor ratio=1, layer 04 has a donor/acceptor ratio<1, and layer 05 consists of 100% acceptor material. In the case of a linear gradient, layer 01 consists, for example, of 100% donor material, layer 02 has a decreasing ratio of donor/acceptor, i.e. the proportion of donor material decreases in a linear manner in the direction of layer 03, and layer 03 consists of 100% acceptor material. The different donor-acceptor ratios can be controlled by means of the deposition rate of each and every material. Such structures can promote the percolation path for charges.

In a further specific embodiment of the present invention, the donor-acceptor heterojunction is configured as an annealed bulk heterojunction; see, for example, Nature 425, 158-162, 2003. The process for producing such a solar cell comprises an annealing step before or after the metal deposition. As a result of the annealing, donor and acceptor materials can separate, which leads to more extended percolation paths.

In a further specific embodiment of the present invention, the organic solar cells are produced by organic vapor phase deposition, either with a flat or a controlled heterojunction architecture. Solar cells of this type are described in Materials, 4, 2005, 37.

The organic solar cells of the invention preferably comprise at least one photoactive region which comprises at least one compound of the formula (I), which is in contact with at least one complementary semiconductor. In addition to compounds of the formula (I), the semiconductor materials listed hereinafter are suitable in principle for use in solar cells according to the invention.

Preferred further semiconductors are fullerenes and fullerene derivatives, preferably selected from C₆₀, C₇₀, C₈₄, phenyl-C₆₁-butyric acid methyl ester ([60]PCBM), phenyl-C₇₁-butyric acid methyl ester ([71]PCBM), phenyl-C₈₄-butyric acid methyl ester ([84]PCBM), phenyl-C61-butyric acid butyl ester ([60]PCBB), phenyl-C61-butyric acid octyl ester ([60]PCBO), thienyl-C61-butyric acid methyl ester ([60]ThCBM) and mixtures thereof. Particular preference is given to C₆₀, [60]PCBM and mixtures thereof. Preference is given to those fullerenes which are vaporizable, for example C60 or C70. Fullerenes and fullerene derivatives in combination with at least one compound of the formula (I) usually act as acceptors.

Suitable further semiconductors are perylendiimides different from the compounds of formula (I). Suitable are e.g. perylendiimides of the formula

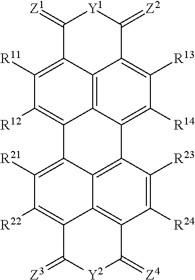

in which

the R¹¹, R¹², R¹³, R¹⁴, R²¹R²², R²³ and R²⁴ radicals are each independently hydrogen, halogen or groups other than halogen,

Y¹ is O or NR^{a} where R^{a} is hydrogen or an organyl radical,

Y² is O or NR^{b} where R^{b} is hydrogen or an organyl radical,

Z¹, Z², Z³ and Z⁴ are each O,

where, in the case that Y¹ is NR^{a}, one of the Z¹ and Z² radicals may also be NR^{c}, where the R^{a} and R^{c} radicals together are a bridging group having 2 to 5 atoms between the flanking bonds, and

where, in the case that Y² is NR^{b}, one of the Z³ and Z⁴ radicals may also be NR^{d}, where the R^{b} and R^{d} radicals together are a bridging group having 2 to 5 atoms between the flanking bonds.

Suitable perylendiimides are, for example, described in WO 2007/074137, WO 2007/093643 and WO 2007/116001, to which reference is made here.

Perylendiimides in combination with at least one compound of the formula (I) may act as donors or acceptors, depending inter alia on the substituents of the perylene diimides.

Further suitable semiconductors are thiophene compounds. These are preferably selected from thiophenes, oligothiophenes and substituted derivatives thereof. Suitable oligothiophenes are quaterthiophenes, quinquethiophenes, sexithiophenes, α,ω-di(C₁-C₈)-alkyloligothiophenes, such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Further thiophene compounds suitable as semiconductors are preferably selected from compounds like

α,α′bis(2,2-dicyanovinyl)quinquethiophene (DCV5T),

(3-(4-octylphenyl)-2,2′-bithiophene) (PTOPT),

and acceptor-substituted oligothiophenes as described in WO 2006/092124.

Thiophene compounds in combination with at least one compound of the formula (I) usually act as donors.

Further semiconductors suitable as donors are merocyanines as described in WO 2010/049512.

All aforementioned semiconductors may be doped. The conductivity of semiconductors can be increased by chemical doping techniques using dopants. An organic semiconductor material may be doped with an n-dopant which has a HOMO energy level which is close to or higher than the LUMO energy level of the electron-conducting material. An organic semiconductor material may also be doped with a p-dopant which has a LUMO energy level which is close to or higher than the HOMO energy level of the hole-conducting material. In other words, in the case of n-doping an electron is released from the dopant, which acts as the donor, whereas in the case of p-doping the dopant acts as an acceptor which accepts an electron.

Suitable dopants for the compounds (I) according to the invention and for p-semiconductors in general are, for example, selected from WO₃, MoO₃, 2,3,5,6-tetrafluoro-7,7,8,8-tetracyanoquinodimethane (F₄-TCNQ), 3,6-difluoro-2,5,7,7,8,8-hexacyanoquinodimethane, dichlorodicyanoquinone (DDQ) or tetracyanoquinodimethane (TCNQ). A preferred dopant is 3,6-difluoro-2,5,7,7,8,8-hexacyanoquinodimethane.

Further suitable dopants are, for example, selected from Cs₂CO₃, LiF, Pyronin B (PyB), rhodamine derivatives, cobaltocenes, etc. Preferred dopants are Pyronin B and rhodamine derivatives, especially rhodamine B.

The dopants are typically used in an amount of up to 10 mol %, preferably up to 5 mol %, based on the amount of the semiconductor to be doped.

The invention further provides an electroluminescent (EL) arrangement comprising an upper electrode, a lower electrode, wherein at least one of said electrodes is transparent, an electroluminescent layer and optionally an auxiliary layer, wherein the electroluminescent arrangement comprises at least one compound of the formula I as defined above. An EL arrangement is characterized by the fact that it emits light when an electrical voltage is applied with flow of current. Such arrangements have been known for a long time in industry and technology as light-emitting diodes (LEDs). Light is emitted on account of the fact that positive charges (holes) and negative charges (electrons) combine with the emission of light. In the sense of this application the terms electroluminescing arrangement and organic light-emitting diode (OLEDs) are used synonymously. As a rule, EL arrangements are constructed from several layers. At least on of those layers contains one or more organic charge transport compounds. The layer structure is in principle as follows:

1. Carrier, substrate

2. Base electrode (anode)

3. Hole-injecting layer

4. Hole-transporting layer

5. Light-emitting layer

6. Electron-transporting layer

7. Electron-injecting layer

8. Top electrode (cathode)

9. Contacts

10. Covering, encapsulation.

This structure represents the most general case and can be simplified by omitting individual layers, so that one layer performs several tasks. In the simplest case an EL arrangement consists of two electrodes between which an organic layer is arranged, which fulfils all functions, including emission of light. The structure of organic light-emitting diodes and processes for their production are known in principle to those skilled in the art, for example from WO 2005/019373. Suitable materials for the individual layers of OLEDs are disclosed, for example, in WO 00/70655. Reference is made here to the disclosure of these documents. In principle OLEDs according to the invention can be produced by methods known to those skilled in the art. In a first embodiment, an OLED is produced by successive vapor deposition of the individual layers onto a suitable substrate. For vapor deposition, it is possible to use customary techniques such as thermal evaporation, chemical vapor deposition and others. In an alternative embodiment, the organic layers may be coated from solutions or dispersions in suitable solvents, for which coating techniques known to those skilled in the art are employed.

Suitable as substrate 1 are transparent carriers, such as glass or plastics films (for example polyesters, such as polyethylene terephthalate or polyethylene naphthalate, polylcarbonate, polyacrylate, polysulphone, polyimide foil). Suitable as transparent and conducting materials are a) metal oxide, for example indium-tin oxide (ITO), tin oxide (NESA), etc. and b) semi-transparent metal films, for example Au, Pt, Ag, Cu, etc.

The compounds of the formula (I) preferably serve as a charge transport material (electron conductor). Thus, at least one compound of the formula I as defined above is preferably used in a hole-injecting layer, hole transporting layer or as part of a transparent electrode.

In the EL applications according to the invention low molecular weight or oligomeric as well as polymeric materials may be used as light-emitting layer 5. The substances are characterized by the fact that they are photoluminescing. Accordingly, suitable substances are for example fluorescent dyes and fluorescent products that are forming oligomers or are incorporated into polymers. Examples of such materials are coumarins, perylenes, anthracenes, phenanthrenes, stilbenes, distyryls, methines or metal complexes such as Alq₃ (tris(8-hydroxyquinolinato)aluminium), etc. Suitable polymers include optionally substituted phenylenes, phenylene vinylenes or polymers with fluorescing segments in the polymer side chain or in the polymer backbone. A detailed list is given in EP-A-532 798. Preferably, in order to increase the luminance, electron-injecting or hole-injecting layers (3 and/or 7) can be incorporated into the EL arrangements. A large number of organic compounds that transport charges (holes and/or electrons) are described in the literature. Mainly low molecular weight substances are used, which are for example vacuum evaporated in a high vacuum. A comprehensive survey of the classes of substances and their use is given for example in the following publications: EP-A 387 715, U.S. Pat. No. 4,539,507, U.S. Pat. No. 4,720,432 and U.S. Pat. No. 4,769,292. A preferred material is PEDOT (poly-(3,4-ethylenedioxythiophene)) which can also be employed in the transparent electrode of the OLEDs.

As a result of the inventive use of the compounds (I), it is possible to obtain OLEDs with high efficiency. The inventive OLEDs can be used in all devices in which electroluminescence is useful. Suitable devices are preferably selected from stationary and mobile visual display units. Stationary visual display units are, for example, visual display units of computers, televisions, visual display units in printers, kitchen appliances and advertising panels, illuminations and information panels. Mobile visual display units are, for example, visual display units in cell phones, laptops, digital cameras, vehicles and destination displays on buses and trains. Moreover, the compounds (I) may be used in OLEDs with inverse structure. The compounds (I) in these inverse OLEDs are in turn preferably used in the light-emitting layer. The structure of inverse OLEDs and the materials typically used therein are known to those skilled in the art.

Before they are used as charge transport materials or exciton transport materials, it may be advisable to subject the compounds of the formula (I) to a purification process. Suitable purification processes comprise conventional column techniques and conversion of the compounds of the formula (I) to the gas phase. This includes purification by sublimation or PVD (physical vapor deposition).

The invention is illustrated in detail with reference to the following nonrestrictive examples.

### EXAMPLES

### I.) Preparation of Compounds of the General Formula I

### Example 1

### N,N′-Bis(1-heptyloctyl)-2,5,10,13-tetrakis[4,4,5,5-tetramethyl-1,3,2-dioxaborolan-2-yl]terrylene-3,4:11,12-tetracarboxylic acid diimide

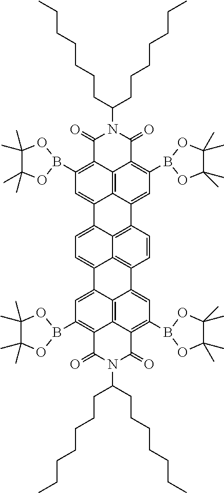

N,N′-Bis(1-heptyloctyl)-terrylene-3,4:11,12-tetracarboxylic acid diimide (0.80 g, 0.86 mmol) and bis(pinacolato)diboron (1.74 g, 6.84 mmol) were mixed together and dissolved in 60 mL anhydrous toluene and 2 mL anhydrous toluene and 2 mL anhydrous acetone. Argon was bubbled through the solution for 30 minutes. RuH₂(CO)(PPh₃)₃ (0.40 g, 0.43 mmol) was added to the mixture and the reaction was heated at 140° C. for 24 hours. After cooling the system to room temperature, the solvent was evaporated and to the remaining liquid were added 400 mL of methanol. The solid was filtered, dissolved in dichloromethane and precipitated once again in methanol. The title compound was obtained as dark blue solid with 60% yield (840 mg, 0.58 mmol).

¹H NMR (500 MHz, CD₂Cl₂) δ 8.74 (s, 4H), 8.53 (s, 4H), 5.19-4.99 (m, 2H), 2.32-2.15 (m, 4H), 1.95-1.75 (m, 4H), 1.39-1.15 (m, 40H), 0.85 (t, J=6.9 Hz, 12H).

¹³C NMR (126 MHz, 393 K, C₂Cl₄D₂) δ 165.49, 133.93, 130.97, 128.95, 128.12, 126.02, 125.70, 124.13, 123.82, 120.24, 84.12, 54.50, 32.44, 31.57, 29.19, 28.82, 26.61, 24.80, 22.25, 13.60.

FD Mass Spectrum (8 kV): m/z=1438.1 (100%) [M+] (Calc. 1438.9)

UV-vis (in chloroform): λₘₐₓ (∈ [M⁻¹cm⁻¹]): 663 nm (1.30×10⁵ M⁻¹cm⁻¹), 608 nm (6.57×10⁴ M⁻¹cm⁻¹), 563 nm (2.11×10⁴ M⁻¹cm⁻¹).

Fluorescence (in chloroform, λₑₓ=632 nm): 680 nm, 739 nm. φ_{F}: 1.00.

### Example 2

### N,N′-Bis(1-heptyloctyl)-2,5,10,13-tetrabromo-terrylene-3,4:11,12-tetracarboxylic acid diimide

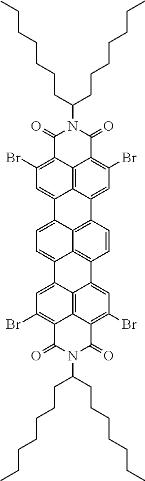 [SMILES: CCCCCCCC(CCCCCCC)N1C(=O)c2c(Br)cc3c4ccc5c6cc(Br)c7c8c(c(Br)cc(c9ccc(c%10cc(Br)c(c2c3%10)C1=O)c4c59)c86)C(=O)N(C(CCCCCCC)CCCCCCC)C7=O]

N,N′-Bis(1-heptyloctyl)-2,5,10,13-tetrakis[4,4,5,5-tetramethyl-1,3,2-dioxaborolan-2-yl]terrylene-3,4:11,12-tetracarboxylic acid diimide (0.40 g, 0.28 mmol) from example 1 and CuBr₂ (0.75 g, 3.33 mmol) were suspended in 100 mL of a 8/1/1 mixture of dioxane/methanol/water and heated at 120° C. for 12 hours in a sealed vessel. The reaction mixture was then cooled to ambient temperature, poured in 0.1 M HCl and filtered. The desired compound was obtained as a blue solid after column chromatography (silica, toluene) in 78% yield (0.27 g, 0.22 mmol).

¹H NMR (250 MHz, CD₂Cl₂) δ 8.41 (s, 4H), 8.10 (s, 4H), 5.25-5.04 (m, 2H), 2.41-2.10 (m, 4H), 2.10-1.85 (m, 4H), 1.30 (m, 40H), 0.87 (t, J=6.6 Hz, 12H).

¹³C NMR (126 MHz, CD₂Cl₂) δ 161.25, 133.93, 132.88, 129.56, 129.49, 128.85, 127.46, 124.37, 124.12, 120.27, 56.25, 32.90, 32.49, 30.21, 29.92, 27.93, 23.29, 14.51.

FD/MS (8 kV): m/z=1251.9 (100%) [M+].

UV-vis (in chloroform): λₘₐₓ (∈ [M⁻¹cm⁻¹]): 640 nm (1.52×10⁵ M⁻¹cm⁻¹), 588 nm (8.23×10⁴ M⁻¹cm⁻¹), 545 nm (2.69×10⁴ M⁻¹cm⁻¹).

Fluorescence (in toluene, λₑₓ=521 nm): 654 nm, 713 nm. φ_{F}: 0.89.

Elem. Anal.: theoretical: C, 61.45%; H, 5.64%; N, 2.24%; experimental: C, 61.28%; H, 5.92%; N, 2.40%.

### Example 3

### N,N′-Bis(1-heptyloctyl)-2,5,10,13-tetracyano-terrylene-3,4:11,12-tetracarboxylic acid diimide

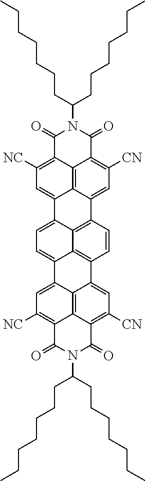 [SMILES: CCCCCCCC(CCCCCCC)N1C(=O)c2c(C#N)cc3c4ccc5c6cc(C#N)c7c8c(c(C#N)cc(c9ccc(c%10cc(C#N)c(c2c3%10)C1=O)c4c59)c86)C(=O)N(C(CCCCCCC)CCCCCCC)C7=O]

70 mg (56 μmol) of the compound from example 1 and copper(I) cyanide (0.10 g, 1.12 mmol) were mixed and suspended in 20 mL anhydrous of DMF under argon atmosphere. The reaction mixture was heated to 130° C. and held at this temperature for 30 minutes. After cooling the reaction mixture to room temperature, 40 mL of a saturated solution of ammonium iron(II) sulfate were added and the mixture was heated to 60° C. for 2 hours. After cooling to room temperature the solution was extracted with chloroform (3×80 mL), the organic phases were collected, extracted with brine (3×80 mL) and dried over magnesium sulfate. Successively chloroform was evaporated and the solid remaining precipitating from the solution was filtered and purified via column chromatography (silica, dichloromethane). The desired product was obtained as a dark blue solid with 42% yield (24 mg, 24 μmol).

¹H NMR (250 MHz, C₂D₂Cl₄) δ 8.89 (s, 4H), 8.77 (s, 4H), 5.19 (m, 2H), 2.20 (m, 4H), 1.98 (m, 4H), 1.25 (m, 40H), 0.85 (m, 12H).

FD/MS (8 kV): m/z=1033.4 (100%) [M+].

UV-vis (in toluene): λₘₐₓ (∈ [M⁻¹cm⁻¹]): 661 nm (1.48×10⁵ M⁻¹cm⁻¹), 607 nm (6.40×10⁴ M⁻¹cm⁻¹), 560 nm (1.79×10⁴ M⁻¹cm⁻¹).

Fluorescence (in chloroform, λₑₓ=632 nm): 680 nm, 739 nm. φ_{F}: 1.00.

### II.) Method for Determining the Transistor Characteristics

### Example 4

All FETs were fabricated employing the bottom-gate, bottom-contact architecture. The 200 nm thick SiO₂ dielectric covering the highly doped Si acting as the gate electrode was functionalized with hexamethyldisilazane (HMDS) to minimize interfacial trapping sites. Thin films of the semiconductor were deposited by drop casting 4 mg mL⁻¹ chloroform solution on FET at room temperature, followed by annealing at 150° C. for 1 h. The channel lengths and widths are 20 and 1400 μm, respectively. All the electrical measurements (using Keithley 4200 SCS) are performed in air.

| Semiconductor | Mobility/cm²/Vs | On/Off Ratio |
|---|---|---|
| Example 3 | 10⁻³ | 1 × 10² |

1: A compound of the general formula I 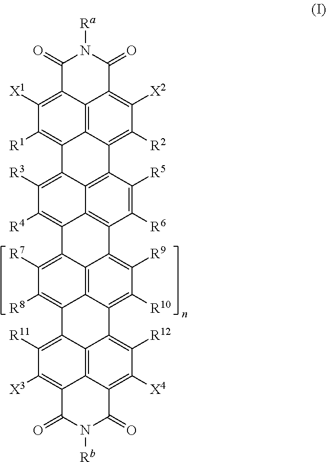
wherein
n is 0 or 1,
R^{a} and R^{b} are independently selected from the group consisting of hydrogen, and in each case unsubstituted or substituted alkyl, alkenyl, alkadienyl, alkynyl, cycloalkyl, bicycloalkyl, cycloalkenyl, heterocycloalkyl, aryl and heteroaryl,
X¹, X², X³ and X⁴ are independently selected from the group consisting of F, Cl, Br, I, CN and B(OR^{c})₂,
wherein R^{c} is selected from the group consisting of, in each case unsubstituted or substituted, alkyl, cycloalkyl and aryl, or wherein two radicals R^{c} may together form a divalent bridging group selected from, in each case unsubstituted or substituted, C₂-C₁₀-alkylene, C₃-C₆-cycloalkylene and C₆-C₁₄-arylene, wherein C₂-C₁₀-alkylene, C₃-C₆-cycloalkylene and C₆-C₁₄-arylene optionally comprise one or more identical or different C₁-C₁₂-alkyl radicals,
R¹, R², R³, R⁴, R⁵, R⁶, R¹¹ and R¹² and, if present, R⁷, R⁸, R⁹ and R¹⁰ are independently selected from the group consisting of hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from the group consisting of hydrogen, alkyl, cycloalkyl, heterocycloalkyl, aryl and hetaryl,
and, in each case unsubstituted or substituted, alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino, and (dihetaryl)amino.

2: The compound according to claim 1, wherein a radical R^{a}, R^{b}, or both is selected from the group consisting of hydrogen, linear C₁-C₃₀-alkyl, branched C₃-C₃₀-alkyl, perfluoro-C₁-C₃₀-alkyl, 1H,1H-perfluoro-C₂-C₃₀-alkyl, 1H,1H,2H,2H-perfluoro-C₃-C₃₀-alkyl, [Si(C₁-C₈-alkyl)(O—Si(C₁-C₈-alkyl)₃)₂]-C₁-C₁₂-alkyl, [Si(phenyl)(O—Si(C₁-C₈-alkyl)₃)₂]-C₁-C₁₂-alkyl, [Si(C₁-C₈-alkyl)(O—Si(C₁-C₈-alkyl)₃)₂]-phenylene [Si(phenyl)(O—Si(C₁-C₈-alkyl)₃)₂]-phenylene, a radical of the formula A.1, a radical of the formula A.2 and a radical of the formula A.3 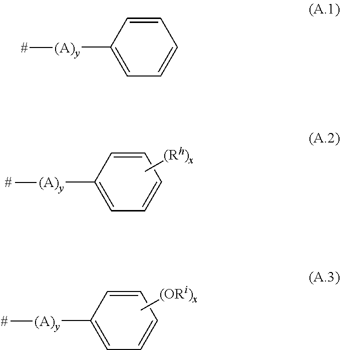
where
# represents a bonding side to a nitrogen atom,
A, where present, is a C₁-C₁₀-alkylene group which may be interrupted by one or more nonadjacent groups selected from —O—, —S—, or both,
Y is 0 or 1,
R^{h} is independently selected from the group consisting of C₁-C₃₀-alkyl, C₁-C₃₀-fluoroalkyl, fluorine, chlorine, bromine, NE¹E², nitro and cyano, where E¹ und E² are each independently selected from the group consisting of hydrogen, alkyl, cycloalkyl, heterocycloalkyl, aryl and hetaryl,
Rⁱ is independently selected from a C₁-C₃₀-alkyl,
x in formulae A.2 and A.3 is 1, 2, 3, 4 or 5.

3: The compound according to claim 1, wherein a radical R^{a}, R^{b}, or both is selected from a radical of the general formula (II) 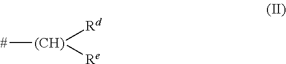
in which
# is a bonding site, and
R^{d} and R^{e} are independently selected from a C₁- to C₂₈-alkyl, where the sum of the carbon atoms of the R^{d} and R^{e} radicals is an integer from 2 to 29.

4: The compound according to claim 1, wherein R^{a} and R^{b} are the same.

5: The compound according to claim 1, wherein X¹, X², X³ and X⁴ are the same.

6: The compound according to claim 1, wherein R¹, R², R³, R⁴, R⁵, R⁶, R¹¹ and R¹² and, if present, R⁷, R⁸, R⁹ and R¹⁰ are all hydrogen.

7: A process for the preparation of a compound of the formula I, 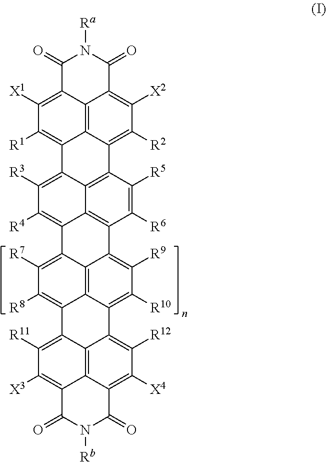
wherein
n is 0 or 1,
R^{a} and R^{b} are independently selected from the group consisting of hydrogen, and in each case unsubstituted or substituted alkyl, alkenyl, alkadienyl, alkynyl, cycloalkyl, bicycloalkyl, cycloalkenyl, heterocycloalkyl, aryl and heteroaryl,
X¹, X², X³ and X⁴ are independently selected from the group consisting of F, Cl, Br, I, CN and B(OR^{c})₂,
wherein R^{e} is selected from the group consisting of, in each case unsubstituted or substituted, alkyl, cycloalkyl and aryl, or wherein two radicals R^{c} may together form a divalent bridging group selected from, in each case unsubstituted or substituted, C₂-C₁₀-alkylene, C₃-C₆-cycloalkylene and C₆-C₁₄-arylene, wherein C₂-C₁₀-alkylene, C₃-C₆-cycloalkylene and C₆-C₁₄-arylene optionally comprise one or more identical or different C₁-C₁₂-alkyl radicals,
R¹, R², R³, R⁴, R⁵, R⁶, R¹¹ and R¹² and, if present, R⁷, R⁸, R⁹ and R¹⁰ are independently selected from the group consisting of hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from the group consisting of hydrogen, alkyl, cycloalkyl, heterocycloalkyl, aryl and hetaryl,
and, in each case unsubstituted or substituted, alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino, and (dihetaryl)amino,
comprising
i) reacting a compound of the formula (A) with a boron-containing compound of the formula (B) in the presence of a transition metal comprising catalyst to obtain a compound of the formula (C) 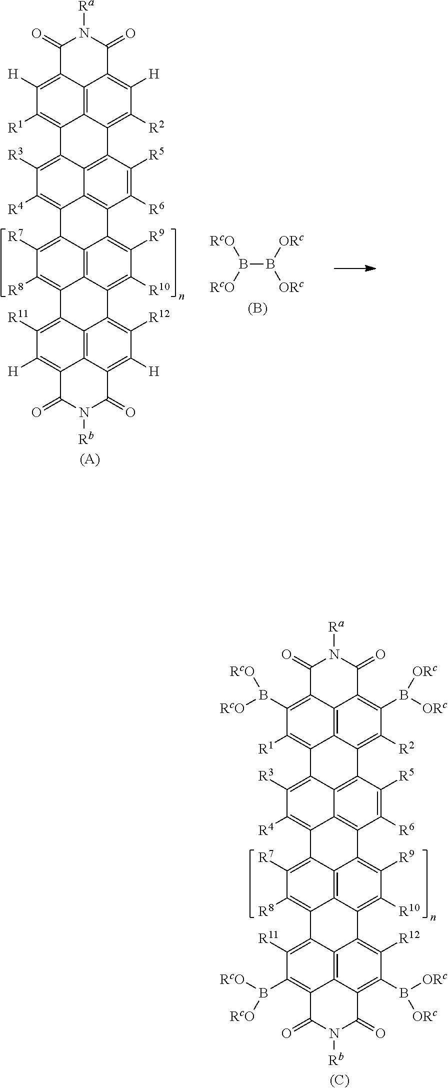
wherein
n, R^{a}, R^{b}, R^{c}, R¹, R², R³, R⁴, R⁵, R⁶, R¹¹ and R¹² and, if present, R⁷, R⁸, R⁹ and R¹⁰, are as defined above, and
ii) optionally reacting the compound of the formula (C) with an F—, Cl—, Br—, I— and/or CN source to obtain a compound of the formula (I).

8: An organic field-effect transistor, comprising a substrate comprising a gate structure, a source electrode and a drain electrode and a compound of the formula I as defined in claim 1 as a semiconductor material.

9: A substrate comprising a plurality of organic field-effect transistors, wherein one or more of the field-effect transistors comprise the compound of the formula I as defined in claim 1.

10: A semiconductor unit comprising the substrate as defined in claim 9.

11: An electroluminescent arrangement comprising an upper electrode, a lower electrode, wherein at least one electrode is transparent, an electroluminescent layer and optionally an auxiliary layer, wherein the electroluminescent arrangement comprises the compound of the formula I as defined in claim 1.

12: The electroluminescent arrangement as claimed in claim 11 further comprising a hole-injecting layer, wherein the hole-injecting layer or the transparent electrode comprises the compound of the formula I.

13: The electroluminescent arrangement as claimed in claim 11 in the form of an organic light-emitting diode (OLED).

14: An organic solar cell comprising the compound of the formula (I) as defined in claim 1.

15: A semiconductor material, comprising the compound of the general formula I as defined in claim 1.

16: The semiconductor material of claim 15, which is present in an organic field-effect transistor.
